(19) United States
(12) Patent Application Publication
(10) Pub. No.: US 2021/0189350 A1
(43) Pub. Date: Jun. 24, 2021

(54) PROCESS FOR MAKING CELL POPULATIONS OF THE HEPATIC LINEAGE FROM ENDODERMAL CELLS AND CELLULAR COMPOSITIONS COMPRISING SAME

(71) Applicant: VALORISATION-HSJ, LIMITED PARTNERSHIP, Montreal (CA)

(72) Inventors: Massimiliano PAGANELLI, Outremont (CA); Claudia RAGGI, Outremont (CA)

(21) Appl. No.: 17/058,299

(22) PCT Filed: May 24, 2019
(86) PCT No.: PCT/CA2019/050705

§ 371 Date: Nov. 24, 2020

Related U.S. Application Data
(60) Provisional application No. 62/676,582, filed on May 25, 2018.

(51) Int. Cl. C12N 5/071

(52) U.S. Cl. CPC C12N 5/0679; C12N 5/067; C12N 2501/15; C12N 2501/115; C12N 2501/12; C12N 2501/155

(57) ABSTRACT

The present disclosure concerns processes as well as additives for differentiating an endodermal cells into a posterior foregut cell, a posterior foregut cell into an hepatic progenitor cell and/or an hepatic progenitor cell into an hepatocyte-like cell. In some embodiments, the process can be conducted in the absence of serum. The hepatocyte-like cell population obtained from this process have a detectable Cyp3A4 activity and/or express a detectable level of albumin and/or of urea. The process can be designed to increase cellular yield.

### CROSS-REFERENCE TO RELATED APPLICATIONS

This application is a national stage application, filed under 35 U.S.C. § 371, of International Patent Application No. PCT/CA2019/050705, filed on May 24, 2019, which claims priority from U.S. provisional application Ser. No. 62/676,582 filed on May 25, 2018 and is herewith incorporated herein in its entirety.

### BACKGROUND

It has proven to be difficult to obtain viable and functional hepatocyte-like cells in high yield, especially when such cells are obtained from differentiating a pluripotent stem cell such as an induced pluripotent stem cell. It has also been proven to be difficult to obtain homogeneous cellular populations in a reproducible manner.

There is thus a need to provide cells from the hepatocyte lineage exhibiting biological activity, especially capable of metabolizing molecules, such as therapeutic agents and/or potential therapeutic agents.

### SUMMARY

The present disclosure concerns processes for differentiating pluripotent cells into viable and functional hepatocyte-like cells by providing or excluding specific additives during culture. The process also for differentiation of pluripotent cells into the endoderm lineage, without favoring, and in some embodiments allowing, the differentiation of pluripotent cells (or resulting differentiated cells) into the mesoderm lineage. The process comprises, to favor differentiation into the endoderm, the activation of the Wnt pathway (to allow Nodal expression) and the TGFβ pathway. The initial transition in the anterior-posterior pattering of the endoderm is started by a combination of Wnt, FGF and BMP signaling at the posterior end of the definitive endoderm. An initial repression of the Wnt pathway in the anterior endoderm coupled with the inhibition of the TGFβ pathway as well as the use of FGF and BMP signaling allows for the expression of Hex (which is required for liver (and pancreas) development). Initial repression of the Wnt signaling is immediately followed by the activation of the same pathway for liver outgrowth. Continued signaling which include FGF, BMP, Wnt and HGF pathways from hepatic mesenchyme and endothelial cells to promote differentiation. For the maturation into hepatocyte-like cells, cytokines, glucocorticoids, HGF and Wnt are beneficial. Cytokines like OSM induce morphological maturation into polarized epithelium.

In a first aspect, the present disclosure provides a process of making posterior foregut cells from endodermal cells. The process comprises contacting the endodermal cells with a first culture medium excluding insulin and comprising a first set of additives under conditions allowing the differentiation of the endodermal cells into the posterior foregut cells. The first set of additives excluding insulin and comprising or consisting essentially of an activator of a bone morphogenetic protein (BMP) signaling pathway; an activator of a fibroblast growth factor (FGF) signaling pathway; an inhibitor of a Wnt signaling pathway; and an inhibitor of a transforming growth factor β (TGFβ) signaling pathway. In an embodiment, the first culture medium comprises serum. In another embodiment, the activator of the BMP signaling pathway is a BMP receptor agonist, for example, BMP4. In another embodiment, the activator of the FGF signaling pathway is a FGF receptor agonist, for example, basic FGF. In a further embodiment, the inhibitor of the Wnt signaling pathway is capable of inhibiting the biological activity of Porcupine, for example, IWP2. In still a further embodiment, the inhibitor of the TGFβ signaling pathway is capable of inhibiting the biological activity of at least one of ALK4, ALK5 or ALK7, for example A83-01. In an embodiment, the endodermal cells express at least one of SOX17, GATA4, FOXA2, CXCR4 or EOMES and/or fail to substantially express c-Kit. As used in the context of the present disclosure, cellular populations of posterior foregut cells “fail to substantially express c-Kit” when less than 3% of the cells are positive for the c-Kit marker. It follows that cells derived from the posterior gut cells, due to their endodermal origin also fail to substantially express c-Kit. In another embodiment, the posterior foregut cells express at least one of SOX2, FOXA1, FOXA2, HNF4a, AFP or albumin. The present disclosure also provides a population of posterior foregut cells obtainable or obtained by the process described herein.

In a second aspect, the present disclosure provides a process for making hepatic progenitor cells from posterior foregut cells. The process comprises contacting the posterior foregut cells with a second culture medium comprising a second set of additives under conditions allowing the differentiation of the posterior foregut cells into the hepatic progenitor cells, wherein the second set of additives comprises or consists essentially of: an activator of an insulin signaling pathway; an activator of a bone morphogenetic protein (BMP) signaling pathway; an activator of a fibroblast growth factor (FGF) signaling pathway; an activator of an hepatocyte growth factor (HGF) signaling pathway; and an activator of a Wnt signaling pathway. In an embodiment, the second culture medium comprises serum. In another embodiment, the activator of the insulin signaling pathway is an insulin receptor agonist, for example, insulin. In another embodiment, the activator of the BMP signaling pathway is a BMP receptor agonist, for example BMP4. In a further embodiment, the activator of the FGF signaling pathway is a FGF receptor agonist, for example, basic FGF. In still another embodiment, the activator of the HGF signaling is a HGF receptor agonist, for example, HGF. In yet a further embodiment, the activator of the Wnt signaling pathway is capable of inhibiting the biological activity of GSK3, for example, CHIR99021. In an embodiment, the posterior foregut cells express at least one of SOX2, FOXA1, FOXA2, HNF4a, AFP or albumin. In another embodiment, the hepatocyte progenitor cells express at least one of α-fetal protein (AFP), albumin (ALB), cytokeratin 7 (CK7), cytokeratin 19 (CK19), SOX9, PDX1, PROX1 or HNF4a. The present disclosure also provides a population of hepatocyte progenitor cells obtainable or obtained by the process described herein.

According to a third aspect, the present disclosure provides a process for making hepatocyte-like cells from hepatic progenitor cells. The process comprises (i) contacting the hepatic progenitor cells with a third culture medium comprising a third set of additives under conditions to obtain cells of the hepatocyte lineage, (ii) contacting the cells of the hepatocyte lineage with a fourth culture medium comprising a fourth set of additives under conditions to obtain immature hepatocyte-like cells and (iii) contacting the immature hepatocyte-like cells with a fifth culture medium excluding cytokines comprising a fifth set of additives under conditions to obtain the mature hepatocyte-like cells. The third set of additives comprises or consists essentially of an activator of an insulin signaling pathway, an activator of a bone morphogenetic protein (BMP) signaling pathway, an activator of a fibroblast growth factor (FGF) signaling pathway, an activator of a hepatocyte growth factor (HGF) signaling pathway, an activator of a Wnt signaling pathway, an inhibitor of a transforming growth factor β (TGFβ) signaling pathway, a cytokine and a glucocorticoid. The fourth set of additives comprises or consists essentially of a cytokine and a glucocorticoid. The fifth set of additives excludes cytokines and comprises or consists essentially of a glucocorticoid. In an embodiment, the fourth, fifth and/or sixth culture medium comprises serum. In another embodiment, the activator of the insulin signaling pathway is an insulin receptor agonist, for example, insulin. In a further embodiment, the activator of the BMP signaling pathway is a BMP receptor agonist, for example, BMP4. In still another embodiment, the activator of the FGF signaling pathway is a FGF receptor agonist, for example, basic FGF. In still a further embodiment, the activator of the HGF signaling pathway is a HGF receptor agonist, for example, HGF. In yet another embodiment, the activator of the Wnt signaling pathway is capable of inhibiting the biological activity of GSK3, for example, CHIR99021. In still a further embodiment, the inhibitor of the TGFβ signaling pathway is capable of inhibiting the biological activity of at least one of ALK4, ALK5 or ALK7, for example, A83-01. In another embodiment, the cytokine is oncostatin M (OSM). In another embodiment, the glucocorticoid is dexamethasone. In still a further embodiment, the hepatic progenitor cells express at least one of α-fetal protein (AFP), albumin (ALB), cytokeratin 7 (CK7), cytokeratin 19 (CK19), SOX9, PDX1, PROX1 or HNF4a. In still a further embodiment, the immature hepatocyte-like cells and/or the mature hepatocyte-like cells express at least one of α-fetal protein (AFP), albumin (ALB), ASGR1, HNF4a or SOX9. In an embodiment, the mature hepatocyte-like cells have a detectable Cyp3A4 activity, express a detectable level of albumin and/or of urea. The present disclosure also provides a population of hepatocyte-like cells obtainable or obtained by the process of described herewith.

According to a fourth aspect, the present disclosure provides a process for making hepatic progenitor cells from endodermal cells. The process comprises or consists essentially of (a) performing the process described herein to obtain posterior foregut cells or providing the population of posterior foregut cells described herein; and (b) submitting the posterior foregut cells to the process described herein to obtain the hepatic progenitor cells. The present disclosure also provides a population of hepatic progenitor cells obtainable or obtained by the process described herein.

According to a fifth aspect, the present disclosure provides a process for making hepatocyte-like cells from hepatic progenitor cells. The process comprises or consists essentially of (a) performing the process described herein to obtain hepatic progenitor cells or providing the population of hepatic progenitor cells described herein; and (b) submitting the hepatic progenitor cells to the process described herein to obtain the hepatocyte-like cells. The process also provides a population of hepatocyte-like cells obtainable or obtained by the process described herein.

According to a sixth aspect, the present disclosure provides a process for making hepatocyte-like cells from endodermal cells. The process comprises or consists essentially of: (a) optionally performing the process described herein to obtain posterior foregut cells or optionally providing the population of posterior foregut cells described herein; (b) submitting the posterior foregut cells to the process described herein to obtain the hepatic progenitor cells or providing the population of hepatic progenitor cells described herein; and (c) submitting the hepatic progenitor cells to the process described herein to obtain the hepatocyte-like cells. The process also provides a population of hepatocyte-like cells obtainable or obtained by the process described herein.

According to a seventh aspect, the present disclosure provides a process for making an encapsulated liver tissue. The process comprises (a) providing a population of hepatocyte-like cells described herein; (b) combining and culturing, in suspension, the hepatic cells, mesenchymal and optionally endothelial cells so as to obtain at least one liver organoid comprising (i) a cellular core comprising mesenchymal and optionally endothelial cells, wherein the cellular core at least partially covered with hepatocyte-like cells and/or biliary epithelial cells, (ii) having a spherical shape and (iii) having a relative diameter between about 50 and about 500 μm; and (c) at least partially covering the at least one liver organoid with a first biocompatible cross-linked polymer. In an embodiment, the endodermal and hepatocyte-like cells are combined, prior to culturing, at a ratio, of 1:0.2-7. In another embodiment, the hepatic and endothelial cells are combined, prior to culturing, at a ratio, 1:0.2-1. In still another embodiment, at least one of the hepatic, endodermal and endothelial cells is obtained from differentiating a pluripotent cell, such as a pluripotent stem cell. In an embodiment, the endothelial cells are endothelial progenitor cells. In a further embodiment, the process comprises substantially covering the at least one liver organoid with the first biocompatible cross-linked polymer, such as, for example, cross-linked polymer comprises poly(ethylene) glycol (PEG). In another embodiment, the process further comprises at least partially covering, and in some embodiments substantially covering, the first biocompatible cross-linked polymer with a second biocompatible cross-linked polymer. In an embodiment, the first biocompatible cross-linked polymer and/or the second biocompatible cross-linked polymer is at least partially biodegradable. In still another embodiment, the second biocompatible cross-linked polymer comprises poly(ethylene) glycol (PEG). The present disclosure also provides an encapsulated liver tissue obtainable or obtained by the process of described herein.

According to an eight aspect, the present disclosure provides sets of additives as well as culture medium comprising same. In an embodiment, the present disclosure provides a first set of additives described herein as well as first culture medium comprising a first set of additives and excluding an activator of the insulin signaling pathway. In an embodiment, the first culture medium further comprises endodermal cells and/or posterior foregut cells. In another embodiment, the present disclosure provides a second set of additives as described herein as well as a second culture medium comprising a second set of additives. In an embodiment, the second culture medium comprises posterior foregut cells and/or hepatic progenitor cells. In still a further embodiment, the present disclosure provides a third set of additives as described herein as well as a third culture medium comprising a third set of additive. In yet another embodiment, the present disclosure provides a fourth set of additives described herein as well as a fourth culture medium comprising a fourth set of additives. In still another embodiment, the present disclosure provides a fifth set of additives described herein as well as a fifth culture medium comprising a fifth set of additive excluding cytokines. The present disclosure also provides a kit for making posterior foregut cells, hepatic progenitor cells or hepatocyte-like cells. The kit comprises at least one set of additives described herein and/or at least one medium described herein; and instructions for making posterior foregut cells, hepatic progenitor cells or hepatocyte-like cells (for example to perform the process described herein). In some embodiment, the kit further comprises endodermal cells, posterior foregut cells and/or hepatic progenitor cells.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A and 1B illustrate the expression of endoderm-specific genes.

FIG. 1A Upregulation of endoderm-specific genes (FOXA2, SOX17, CXCR4, EOMES, GATA4) in iPSC-derived endodermal cells (DE—dark gray bars) when compared to undifferentiated iPSCs (iPSC—light gray bars) as measured by RT-qPCR. Results are shown as logarithmic fold change in the various genes tested. Data are mean±s.d. N=6 for DE, N=3 for iPSC. *p<0.05 **p<0.01.

FIG. 1B Time course analysis by RT-qPCR of endoderm-specific genes (EOMES, FOXA2, SOX17) expression during iPSC differentiation into endoderm. Results are shown as logarithmic fold change in the various genes tested (identified on the X-axis). Data are mean±s.d. N=3 for all the time points **p<0.01 ***p<0.001 ****p<0.0001.

FIG. 2 provides a representative flow cytometry analysis of iPSC-derived endodermal cells for the FoxA2, Cxcr4, Sox17, Brachyury and c-Kit markers. More than 85% of the cells are triple positive for FoxA2, Cxcr4 and Sox17; 90% of the cells are positive for brachyury; less than 1% of the cells were positive for c-Kit showing the absence of mesodermal cells. Data are mean±s.d. n=4.

FIG. 3 provides a representative immunofluorescence analysis of endodermal markers Sox17, FoxA2 and Cxcr4 in iPSC-derived endodermal cells (bottom panel) and undifferentiated iPSCs (top panel). Inserts show nucleus (DAPI) staining (scale bar 200 μm).

FIG. 4 shows the increased expression of posterior foregut's specific genes in iPSC-derived ventral posterior foregut cells, which give rise to hepatic progenitor cells. Results are shown as fold change in mRNA expression of those genes (FOXA2, SOX2, FOXA1, HNF4a, AFP and albumin (ALB)) in iPSC-derived endodermal cells (DE—dark gray bars), and in iPSC-derived posterior foregut cells (PFG—light gray bars). Data are mean±s.d. n=3 for DE, N=6 for PFG *p<0.05 **p<0.01 ***p<0.001.

FIGS. 5A to 5D illustrate the expression of hepatic specific markers in iPSC-derived hepatic progenitor cells (scale bar 200 μM) as determined by immunofluorescence.

FIG. 5A shows the results obtained for AFP.

FIG. 5B shows the results obtain for albumin.

FIG. 5C shows the results obtained for CK19.

FIG. 5D shows the results obtained for EpCAM.

FIG. 6 provides a representative flow cytometry analysis of the iPSC-derived hepatic progenitor cells (HB—gray bars) in comparison to undifferentiated iPSCs (iPSC—white bars) for pluripotency markers TRA1-60 and Nanog. Data are mean±s.d. n=3.

FIG. 7 shows the expression of hepatoblast and hepatocyte specific genes (albumin (ALB), AFP, CK19, CK7, PDX1, SOX9, PROX1, HNF4a, HHEX) in iPSC-derived hepatic progenitor cells (HB—black bars) as compared to iPSC-derived posterior foregut cells (PFG—light gray bars) as determined by RT-qPCR. Results are shown as logarithmic fold change in the various genes tested (identified on the X-axis). Data are mean±s.d. n=8 for HB, n=3 for PFG **p<0.01.

FIG. 8 shows a time course of cell proliferation during iPSC differentiation into hepatic progenitor cells showing a significant increase in the cell yield. Data are mean±s.d. n=6 for undifferentiated iPSCs (iPSC), n=3 for iPSC-derived endodermal cells (DE), n=6 for iPSC-derived hepatic progenitor cells (HB). **p<0.01.

FIGS. 9A and 9B illustrate the characteristics of iPSC-derived hepatocyte-like cells.

FIG. 9A Representative aspects of iPSC-derived hepatocyte-like cells (HLC) at day 28 (scale bar 1 000 μm for top panel, 200 μm for bottom panel) as determined by light microscopy.

FIG. 9B Expression of hepatic specific markers (FIG. 9B1 AFP, FIG. 9B2 albumin, FIGS. 9B3 and 9B4 CK19) in iPSC-derived hepatic-like cells as determined by immunofluorescence (scale bar 200 μm top and left bottom panels, 100 μm right bottom panel).

FIG. 9B1 shows the results obtained for AFP.

FIG. 9B2 shows the results obtained for albumin.

FIG. 9B3 shows the results obtained for CK19.

FIG. 9B4 shows the results obtained for CK19.

FIGS. 10A and 10B provide the expression of albumin expression in iPSC-derived hepatocyte-like cells. Data are mean±s.d n=4.

FIG. 10A shows the results of a representative flow cytometry.

FIG. 10B shows the results associated analysis of albumin-expressing iPSC-derived hepatocyte-like cells (HLC, from FIG. 10A) showing high homogeneity of albumin expression (98.5% of the gated cells).

FIG. 11 provides the expression of hepatic specific genes (HNF4a, AFP, albumin (ALB), SOX9, ASGPR) in iPSC-derived hepatocyte-like cells (HLC—dark gray bars) as compared to freshly isolated fetal hepatocytes (FPHH—light gray bars) as determined by RT-qPCR. Results are show as the logarithmic fold change in the various genes tested (identified on the X-axis). Data are mean±s.d. n=6 for FPHH, N=10 for HLC **p<0.01; ns=not significant.

FIGS. 12A to 12C show the liver-specific functions of primary human hepatocytes (PHH), human liver cancer cell line (HepG2), non-differentiated iPSC (iPSC), iPSC-derived endodermal cells (DE), iPSC-derived ventral posterior foregut cells (PFG), iPSC-derived hepatic progenitor cells (HB), and iPSC-derived hepatocyte-like cells (HLC).

FIG. 12A shows the comparison of CyP3A4 activity. Results are shown as activity (RLU/1×10⁶ cells) in function of the condition tested. Data are mean±s.d n=10 for PHH, n=3 for HepG2 and iPSC, N=6 for HLC *p<0.05.

FIG. 12B shows the comparison of albumin synthesis. Data are mean±s.d n=3 for iPSC, DE, PFG and HB n=6 for HLC, n=10 for PHH **p<0.01.

FIG. 12C shows the comparison of urea. Data are mean±s.d n=3 for HepG2, n=6 for HLC, n=10 for PHH.

FIG. 13 provides the expression of hepatic specific genes (HNF4a, AFP, albumin (ALB), ASGR1, TAT) in iPSC-derived hepatocyte-like cells (HLC-B, gray bars) as compared to iPSC-derived hepatocyte-like cells (HLC-A, black bars) obtained with standard differentiation protocol, as determined by RT-qPCR. Results are shown as logarithmic fold change in the various genes tested (identified on the X-axis). Data are mean±s.d n=8 for HLC-A n=4 for HLC-B *p<0.05 ***p<0.001 ****p<0.0001.

FIGS. 14A to 14C compare the characteristics of iPSC-derived hepatocyte-like cells (HLC-A, black bars) iPSC-derived hepatocyte-like cells (HLC-B, gray bars).

FIG. 14A shows the comparison of CyP3A4 activity. Results are shown as activity (RLU/1×10⁶ cells) in function of the condition tested. Data are mean±s.d N=4 for HLC-A N=6 for HLC-B **p<0.01.

FIG. 14B shows the comparison of albumin synthesis. Data are mean (μg/1×10⁶ cells/24 h)±s.d N=4 for HLC-A N=6 for HLC-B **p<0.01.

FIG. 14C shows the yield of the cells at the end of the differentiation: a significant increase of the cell number is observed with the new differentiation protocol (light gray bar) while a decrease occurs with the standard differentiation protocol (black bar) in comparison to the amount of undifferentiated iPSCs (white bar) at the beginning of the process. Data are mean±s.d n=3 for HLC-A n=4 for HLC-B *p<0.05.

FIG. 15 provides the measurement by Seahorse of the oxygen consumption rate (OCR) to asses key parameters of mitochondrial function on the iPSC-derived hepatocyte-like cells (HLC) at base line (light grey bars), and after different doses of amiodarone (2, 4, 8, 16 μM—dark gray bars) and acetaminophen (2, 4, 8 mM—black bars). Data are mean±s.d n=6. *p<0.05 **p<0.01 ***p<0.001 ****p<0.0001.

### DETAILED DESCRIPTION

Processes for Making Cells and Compositions Comprising Same

In accordance with the present invention, there is provided a process of differentiating an endodermal cell into a cell of the hepatic lineage capable (e.g., a posterior foregut cell, an hepatic progenitor cell and/or an hepatocyte). The cell of the hepatic lineage can be a cell capable of differentiating into an hepatocyte or being an hepatocyte. The processes of the present disclosure are advantageous because, in some embodiments, they allow the production of more and/or of more biologically potent cells of the hepatic lineage.

In an embodiment, the process can be used to make various cell populations from an endodermal cell. As used in the present disclosure, an “endodermal cell” refers to a cell having the characteristics of a cell from an endoderm. As it is known in the art of embryology, the endoderm is the innermost layer of the three primary germ layers. Cells of the endoderm are generally flattened and are destined to give rise to most of the gastrointestinal tract, respiratory, liver, pancreatic, endocrine and urinary cells. Endodermal cells can be identified by those skilled in the art using various techniques known in the art. For example, endodermal cells can be identified by determining the presence or absence as well as the expression levels of at least one or any combinations of the following genes: SOX17, GATA4, FOXA2, CXCRA and/or EOMES or the polypeptides they encode. In a specific embodiment, the endodermal cell expresses at least two or any combinations of the following genes: SOX17, GATA4, FOXA2, CXCRA and/or EOMES or the polypeptides they encode. In still another embodiment, the endodermal cell can be identified by detecting and optionally measuring the expression of at least three or any combinations of the following genes: SOX17, GATA4, FOXA2, CXCRA and/or EOMES or the polypeptides they encode. In yet another embodiment, the endodermal cell expresses and can be identified by detecting and optionally measuring the expression of at least four or any combinations of the following genes: SOX17, GATA4, FOXA2, CXCRA and/or EOMES. In still another embodiment, the endodermal cell expresses and can be identified by detecting and optionally measuring the expression of the following genes (or their associated polypeptides): SOX17, GATA4, FOXA2, CXCRA and EOMES. In some embodiments, the endodermal cell expresses and can be identified by comparing the level of expression of the following genes or the polypeptides they encode: SOX2, SOX17, GATA4, FOXA2, CXCRA and EOMES with the level of expression of the same genes/polypeptides in an (undifferentiated) stem cell. In specific embodiments, the endodermal cell expresses more of the SOX17, GATA4, FOXA2, CXCR4 and/or EOMES genes or the polypeptides they encode when compared to a corresponding level in the undifferentiated pluripotent (stem) cell.

The endodermal cell can be of any origin, it can especially be derived from a mammal and, in some embodiments from a human.

The endodermal cell can be obtained from a pluripotent cell (for example an embryonic or a pluripotent stem cell) which has been differentiated into an endodermal cell. In some embodiments, the endodermal cell can be obtained by differentiating an induced pluripotent stem cell (iPSC). The pluripotent (stem) cell can be of any origin, it can especially be derived from a mammal and, in some embodiments, from a human. In some embodiments for differentiating the pluripotent (stem) cell into an endodermal cell, the pluripotent (stem) cell can be contacted with a compound capable of activating the Nodal/Activin signaling pathway, for example, a Nodal/Activin receptor agonist such as Activin A. In some additional embodiments, the pluripotent (stem) cell can also be contacted with an activator of the Wnt signaling pathway, for example a Wnt receptor agonist or a compound capable of inhibiting the biological activity of GSK3, such as, for example CHIR99021.

The pluripotent (stem) cell, prior to being differentiated into an endodermal cell, can be contacted with one or more activators of the APELA/ELABELA signaling pathway, for example an agonist of an APELA/ELABELA receptor, such as the APELA/ELABELA polypeptide or a functional fragment (such as those described in U.S. Pat. No. 9,309,314) for inducing, optimizing and maintaining its self-renewal and/or the pluripotency.

The present disclosure provides a first process for making, from an endodermal cell, a posterior foregut cell. The process includes contacting one or more endodermal cells with a first culture medium comprising a first set of additives under conditions so as to allow the differentiation of the endodermal cell into the posterior foregut cell. The first process excludes contacting the cultured cells with an activator of the insulin signaling pathway, such as, for example, insulin. As used in the present disclosure, a “posterior foregut cell” refers to a cell having the biological characteristics of a cell of the posterior foregut. As known in the art of embryology, the posterior foregut is a region of the endoderm from which the liver is subsequently formed. Cells of the posterior foregut are thus capable of further differentiating into the liver, the pancreas, the stomach and part of the small bowel. Posterior foregut cells can be identified by those skilled in the art using various techniques known in the art. For example, posterior foregut cells can be identified by determining the presence or absence as well as the expression levels of at least one of any combinations of the following genes: SOX2, FOXA1, FOXA2, HNF4a, AFP and/or albumin or the polypeptides they encode. In a specific embodiment, the posterior foregut cell expresses at least two of any combinations of the following genes: SOX2, FOXA1, FOXA2, HNF4a, AFP and/or albumin or the polypeptides they encode. In still another embodiment, the posterior foregut cell expresses at least three of any combinations of the following genes: SOX2, FOXA1, FOXA2, HNF4a, AFP and/or albumin or the polypeptides they encode. In yet another embodiment, the posterior foregut cell expresses at least four of any combinations of the following genes: SOX2, FOXA1, FOXA2, HNF4a, AFP and/or albumin or the polypeptides they encode. In yet another embodiment, the posterior foregut cell expresses at least five of any combinations of the following genes: SOX2, FOXA1, FOXA2, HNF4a, AFP and/or albumin or the polypeptides they encode. In yet another embodiment, the posterior foregut cell expresses the following genes: SOX2, FOXA1, FOXA2, HNF4a, AFP and albumin or the polypeptides they encode. In yet another embodiment, the posterior foregut cell expresses and can be identified by detecting and optionally measuring the expression of the following genes (or their corresponding polypeptides): SOX2, FOXA1, FOXA2, HNF4a, AFP and/or albumin. In some embodiments, the posterior foregut cell expresses and can be identified by comparing the level of expression of the following genes or the polypeptides they encode: SOX2, FOXA1, FOXA2, HNF4a, AFP and/or albumin with the level of expression of the same genes/polypeptides in an (undifferentiated) pluripotent (stem) cell or an endodermal cell. In specific embodiments, the posterior foregut cell expresses more of the SOX2, FOXA1, FOXA2, HNF4a, AFP and/or albumin genes or the polypeptides they encode when compared to a corresponding level in the pluripotent (stem) cell or an endodermal cell. In additional embodiment, the posterior foregut cell expresses a higher level of the SOX2 gene or the polypeptide it encodes when compared to a corresponding level in an endodermal cell. In a further embodiment, the posterior foregut cell expresses more of the FOXA1 gene or the polypeptide it encodes when compared to a corresponding level in an endodermal cell. In a further embodiment, the posterior foregut cell expresses more of the FOXA2 gene or the polypeptide it encodes when compared to a corresponding level in an endodermal cell. In a further embodiment, the posterior foregut cell expresses more of the HNF4a gene or the polypeptide it encodes when compared to a corresponding level in an endodermal cell. In a further embodiment, the posterior foregut cell expresses more of the AFP gene or the polypeptide it encodes when compared to a corresponding level in an endodermal cell. In a further embodiment, the posterior foregut cell expresses more of the ALB gene or the albumin polypeptide it encodes when compared to a corresponding level in an endodermal cell.

The posterior foregut cell can be of any origin, it can especially be derived from a mammal and, in some embodiments from a human.

The first culture medium used in the first process can be serum free (e.g., not supplemented with serum). In an alternative embodiment, the first culture medium used in the first process can comprise serum, which can be KnockOut Serum Replacement™ (ThermoFisher Scientific). In an embodiment, the first culture medium comprises between about 0.1 and about 5% (v/v) serum. In still another embodiment, the first culture medium comprises at least about 0.1, 0.2, 0.3, 0.4, 0.5, 0.6, 0.7, 0.8, 0.9, 1, 1.5, 2, 2.5, 3, 3.5, 4, 4.5% or more of serum. In another embodiment, the first culture medium comprises less than about 5, 4.5, 4, 3.5, 3, 2.5, 2, 1.5, 1, 0.9, 0.8, 0.7, 0.6, 0.5, 0.4, 0.3, 0.2% or less of serum. In yet another embodiment, the firs culture medium comprises between about 0.1, 0.2, 0.3, 0.4, 0.5, 0.6, 0.7, 0.8, 0.9, 1, 1.5, 2, 2.5, 3, 3.5, 4 or 4.5% and about 5, 4.5, 4, 3.5, 3, 2.5, 2, 1.5, 1, 0.9, 0.8, 0.7, 0.6, 0.5, 0.4, 0.3 or 0.2% serum. In an embodiment, the first culture medium comprises about 1% serum.

The first culture medium includes a first set of additives which comprises or consists essentially of an activator of a bone morphogenetic protein (BMP) signaling pathway; an activator of a fibroblast growth factor (FGF) signaling pathway; an inhibitor of a Wnt signaling pathway; and an inhibitor of a transforming growth factor β (TGFβ) signaling pathway. The first set of additives excludes an activator of an insulin signaling pathway such as insulin. As used in the context of the present disclosure, the expression “first culture medium consists essentially of a first set of additives” refers to a first culture medium comprising additional additives which are not essential for the differentiation of the endodermal cell into a posterior foregut cell but can nevertheless facilitate the differentiation. These additional additives include, but are not limited to retinoic acid, vitamins and minerals for example.

The first culture medium comprises an activator of a bone morphogenetic protein (BMP) signaling pathway. During development, activators of the BMP signaling pathway are usually being provided by the cardiac mesoderm and favor the differentiation of endodermal cells into posterior foregut cells. As used in the context of the present disclosure, an “activator of a BMP signaling pathway” refers to a compound capable of activating the signaling pathway associated with the binding of a BMP to its cognate receptor (for example BMPR1 and/or BMPR2). Signal transduction the BMP receptors occurs via SMAD and MAP kinase pathways to effect transcription of BMP target genes. The compound can either be an agonist of the BMP receptor (either specific for BMPR1 or BMPR2 or capable of binding and activating both receptors), an activator of a polypeptide known to be activated in the BMP signaling pathway and/or an inhibitor of a polypeptide known to be inhibited in the BMP signaling pathway. Known BMPs include, but are not limited to, BMP1, BMP2, BMP3, BMP4, BMP5, BMP6, BMP7, BMP8a, BMP8b, BMP9, BMP10, BMP11 and BMP15. In an embodiment, the activator is DM3189. In another embodiment, the activator is BMP4 (which can be provided in a recombinant or purified form). BMP4 is a member of the transforming growth factor-β (TGF-β) family binds to two different types of serine-threonine kinase receptors known as BMPR1 and BMPR2. In embodiments in which BMP4 is provided as the activator of the BMP signaling pathway, it can be provided at a concentration of at least about 10, 11, 12, 13, 14, 15, 16, 17, 18, 19, 20, 21, 22, 23, 24, 25, 26, 27, 28, 29 or more ng/mL of the first culture medium. In embodiments in which BMP4 is provided as the activator of the BMP signaling pathway, it can be provided at a concentration of no more than about 30, 29, 28, 27, 26, 25, 24, 23, 22, 21, 20, 19, 18, 17, 16, 15, 14, 13, 12, 11 or less ng/mL of the first culture medium. In embodiments in which BMP4 is provided as the activator of the BMP signaling pathway, it can be provided at a concentration of between about 10, 11, 12, 13, 14, 15, 16, 17, 18, 19, 20, 21, 22, 23, 24, 25, 26, 27, 28 or 29 and about 30, 29, 28, 27, 26, 25, 24, 23, 22, 21, 20, 19, 18, 17, 16, 15, 14, 13, 12 or 11 ng/mL of the first culture medium. In some specific embodiments, BMP4 can be provided at a concentration of about 20 ng/mL of the first culture medium.

The first culture medium also comprises an activator of a fibroblast growth factor (FGF) signaling pathway. During development, activators of the FGF signaling pathway are usually being provided by the cardiac mesoderm and favor the differentiation of endodermal cells into posterior foregut cells. As used in the context of the present disclosure, an “activator of a FGF signaling pathway” refers to a compound capable of activating the signaling pathway associated with the binding of a FGF to its cognate receptor (for example FGFR1, FGFR2, FGFR3 and/or FGFR4). The compound can either be an agonist of the FGF receptor (either specific for FGFR1, FGFR2, FGFR3 and/or FGFR4 or capable of binding and activating more than one receptor), an activator of a polypeptide known to be activated in the FGF signaling pathway and/or an inhibitor of a polypeptide known to be inhibited in the FGF signaling pathway. Known FGFs include, but are not limited to, FGF1, FGF2, FGF3, FGF4, FGF5, FGF6, FGF7, FGF8a, FGF8b, FGF9, FGF10, FGF11, FGF12, FGF13, FGF14, FGF15/19, FGF16, FGF17, FGF18, FGF20, FGF21, FGF22 and FGF23. In an embodiment, the activator is basic FGF or FGF2 (which can be provided in a recombinant or purified form). FGF2 binds to two different types of receptors known as FGFR2 (also known as CD332) and FGFR3. In embodiments in which basic FGF is provided as the activator of the FGF signaling pathway, it can be provided at a concentration of at least about 1, 2, 3, 4, 5, 6, 7, 8, 9, 10, 11, 12, 13, 14, 15, 16, 17, 18, 19 or more ng/mL of the first culture medium. In embodiments in which basic FGF is provided as the activator of the FGF signaling pathway, it can be provided at a concentration of no more than about 20, 19, 18, 17, 16, 15, 14, 13, 12, 11, 10, 9, 8, 7, 6, 5, 4, 3, 2 or less ng/mL of the first culture medium. In embodiments in which basic FGF is provided as the activator of the FGF signaling pathway, it can be provided at a concentration of between about 1, 2, 3, 4, 5, 6, 7, 8, 9, 10, 11, 12, 13, 14, 15, 16, 17, 18 or 19 and about 20, 19, 18, 17, 16, 15, 14, 13, 12, 11, 10, 9, 8, 7, 6, 5, 4, 3, 2 ng/ml of the first culture medium. In some specific embodiments, basic FGF can be provided at a concentration of about 5 ng/mL of the first culture medium.

The first culture medium further comprises an inhibitor of a Wnt signaling pathway. The presence of the inhibitor of the Wnt signaling pathway, in combination with an inhibitor of the TGFβ signaling pathway, favors the expression of the HEX and PROX1 genes which encode polypeptides required for liver development. As used in the context of the present disclosure, an “inhibitor of a Wnt signaling pathway” refers to a compound capable of inhibiting the signaling pathway associated with the binding of a Wnt protein ligand to its cognate Frizzled receptor (for example FZD1, FZD2, FZD3, FZD4, FZDS, FZD6, FZD7, FZD8, FZD9 or FZD10). The family of Frizzled receptors are G protein-coupled receptor proteins. The compound can either be an antagonist of the Frizzled receptor (either specific for FZD1, FZD2, FZD3, FZD4, FZDS, FZD6, FZD7, FZD8, FZD9 or FZD10 or capable of binding and inhibiting more than one receptor), an inhibitor of a polypeptide known to be activated in the Wnt signaling pathway and/or an activator of a polypeptide known to be inhibited in the Wnt signaling pathway. Known Wnt proteins include, but are not limited to, WNT1, WNT2, WNT2B, WNT3, WNT3A, WNT4, WNTSA, WNTSB, WNT6, WNT7A, WNT7B, WNT8A, WNT8B, WNT9A, WNT9B, WNT10A, WNT10B, WNT11 and WNT16. In an embodiment, the inhibitor is capable of inhibiting the biological activity of one or more Frizzled receptors. In another embodiment, the inhibitor is capable of inhibiting the biological activity of the Porcupine protein. For example, the inhibitor capable of inhibiting the biological activity of the Porcupine protein can be IWP2. In an embodiment in which IWP2 is used as the inhibitor of the Wnt signaling pathway, it can be provided at a concentration of at least 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 μM or more in the first culture medium. In an embodiment in which IWP2 is used as the inhibitor of the Wnt signaling pathway, it can be provided at a concentration of no more than 10, 9.5, 9, 8.5, 8, 7.5, 7, 6.5, 6, 5.5, 5, 4.5, 4, 3.5, 3, 2.5, 2, 1.5, 1, 0.9, 0.8, 0.7, 0.6, 0.5, 0.4, 0.3, 0.2 μM or less in the first culture medium. In an embodiment in which IWP2 is used as the inhibitor of the Wnt signaling pathway, it can be provided at a concentration between about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9 or 9.5 and about 10, 9.5, 9, 8.5, 8, 7.5, 7, 6.5, 6, 5.5, 5, 4.5, 4, 3.5, 3, 2.5, 2, 1.5, 1, 0.9, 0.8, 0.7, 0.6, 0.5, 0.4, 0.3 or 0.2 μM in the first culture medium. In an embodiment in which IWP2 is used as the inhibitor of the Wnt signaling pathway, it can be provided at a concentration of about 4 μM in the first culture medium.

The first culture medium further comprises an inhibitor of a transforming growth factor β (TGFβ) signaling pathway. The presence of the inhibitor of the TGFβ signaling pathway, in combination with the presence of an inhibitor of the Wnt signaling pathway, favors the expression of the HEX and PROX1 genes which encode polypeptides required for liver development. As used in the context of the present disclosure, an “inhibitor of a TGFβ signaling pathway” refers to a compound capable of inhibiting the signaling pathway associated with the binding of TGFβ to its cognate receptor. The family of TGFβ receptors mediate signalization via the SMAD proteins. The compound can either be an antagonist of the TGFβ receptor, an inhibitor of a polypeptide known to be activated in the TGFβ signaling pathway and/or an activator of a polypeptide known to be inhibited in the TGFβ signaling pathway. Known TGFβ proteins include, but are not limited to, TGFB1, TGFB2, TGFB3 and TGFB4. In an embodiment, the inhibitor is capable of inhibiting the biological activity of at least one of the ALK4, ALK5 or ALK7 polypeptides. In some embodiments, the inhibitor is capable of inhibiting the biological activity of the ALK4, ALK5 and ALK7 polypeptides. For example, the inhibitor capable of inhibiting the biological activity of the ALK4, ALK5 and ALK7 polypeptides can be A83-01. Alternatively or in combination, the inhibitor can be SB431542 and/or LY364947. In an embodiment in which A83-01 is used as the inhibitor of the TGFβ signaling pathway, it can be provided at a concentration of at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5 μM or more in the first culture medium. In an embodiment in which A83-01 is used as the inhibitor of the TGFβ signaling pathway, it can be provided at a concentration of no more than 5, 4.5, 4., 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 μM or less in the first culture medium. In an embodiment in which A83-01 is used as the inhibitor of the TGFβ signaling pathway, it can be provided at a concentration between 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4 or 4.5 and about 5, 4.5, 4., 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3 or 0.2 μM in the first culture medium. In an embodiment in which A83-01 is used as the inhibitor of the TGFβ signaling pathway, it can be provided at a concentration of about 1 μM in the first culture medium.

The first culture medium remains in contact with the endodermal cells and the posterior foregut cells for at least one day or more until differentiation occurs. If the first medium is intended to be in contact with the cultured cells for more than one day, it can be changed daily. In some embodiments of the process of the present disclosure, the first culture medium remains in contact at least 1, 2, 3, 4 or more days with the cultured cells. In another embodiment, the first culture medium remains in contact no more than 5, 4, 3, 2 or less days with the cultured cells. In still another embodiment, the first culture medium remains in contact at least 1, 2, 3, 4 or more days and no more than 5, 4, 3, 2 or less days with the cultured cells. In yet another embodiment, the first culture medium remains in contact between about 1 and 5 days with the cultured cells.

The use of the first culture medium with endodermal cells allows the differentiation of endodermal cells into posterior foregut cells. Therefore, the present disclosure provides a population of posterior foregut cells obtained from the process described herein. In the population of posterior foregut cells of the present disclosure, the majority of the cells are considered posterior foregut cells and, in some embodiments, can include some endodermal cells.

The present disclosure provides a second process for making, from a posterior foregut cell, an hepatic progenitor cell (also referred to herein as an hepatoblast). The process includes contacting one or more posterior foregut cell cells with a second culture medium comprising a second set of additives under conditions so as to allow the differentiation of the posterior foregut cell into the posterior foregut cell. The posterior foregut cells used in the second process can be obtained from performing the first process.

As used in the present disclosure, an “hepatic progenitor cell” or an “hepatoblast” refers to a bi-potent progenitor cell capable of differentiating either in cholangiocytes and hepatocytes. Hepatic progenitor cells can be identified by those skilled in the art using various techniques known in the art. For example, hepatic progenitor cells can be identified by determining the presence or absence as well as the expression levels of at least one or any combinations of the following genes: α-fetal protein (AFP), albumin (ALB), cytokeratin 7 (CK7), cytokeratin 19 (CK19), SOX9, PDX1, PROX1, EpCAM, HHEX gene and/or HNF4a or the polypeptides they encode. In a specific embodiment, the an hepatic progenitor cell expresses at least one or any combinations of the following genes: α-fetal protein (AFP), albumin (ALB), cytokeratin 7 (CK7), cytokeratin 19 (CK19), SOX9, PDX1, PROX1, EpCAM, HHEX or HNF4a or the polypeptides they encode. In still another embodiment, the hepatic progenitor cell expresses at least one of the following genes: α-fetal protein (AFP), albumin (ALB), cytokeratin 7 (CK7), cytokeratin 19 (CK19), SOX9, PDX1, PROX1, EpCAM, HHEX or HNF4a or the polypeptides they encode. In yet another embodiment, the hepatic progenitor cell expresses at least two of any combination of the following genes: α-fetal protein (AFP), albumin (ALB), cytokeratin 7 (CK7), cytokeratin 19 (CK19), SOX9, PDX1, PROX1, EpCAM, HHEX and/or HNF4a or the polypeptides they encode. In yet another embodiment, the hepatic progenitor cell expresses at least three of any combinations of the following genes: α-fetal protein (AFP), albumin (ALB), cytokeratin 7 (CK7), cytokeratin 19 (CK19), SOX9, PDX1, PROX1, EpCAM, HHEX and/or HNF4a or the polypeptides they encode. In yet another embodiment, the hepatic progenitor cell expresses at least four of any combinations of the following genes: α-fetal protein (AFP), albumin (ALB), cytokeratin 7 (CK7), cytokeratin 19 (CK19), SOX9, PDX1, PROX1, EpCAM, HHEX and/or HNF4a or the polypeptides they encode. In yet another embodiment, the hepatic progenitor cell expresses at least five of any combinations of the following genes α-fetal protein (AFP), albumin (ALB), cytokeratin 7 (CK7), cytokeratin 19 (CK19), SOX9, PDX1, PROX1, EpCAM, HHEX and/or HNF4a. In yet another embodiment, the hepatic progenitor cell expresses at least six or more polypeptides encoded by any combinations of the following genes: α-fetal protein (AFP), albumin (ALB), cytokeratin 7 (CK7), cytokeratin 19 (CK19), SOX9, PDX1, PROX1, EpCAM, HHEX and/or HNF4a. In yet another embodiment, the hepatic progenitor cell expresses at least seven or more polypeptides encoded by any combinations of the following genes: α-fetal protein (AFP), albumin (ALB), cytokeratin 7 (CK7), cytokeratin 19 (CK19), SOX9, PDX1, PROX1, EpCAM, HHEX and/or HNF4a. In yet another embodiment, the hepatic progenitor cell expresses at least eight or more polypeptides encoded by any combinations of the following genes: α-fetal protein (AFP), albumin (ALB), cytokeratin 7 (CK7), cytokeratin 19 (CK19), SOX9, PDX1, PROX1, EpCAM, HHEX and/or HNF4a. In yet another embodiment, the hepatic progenitor cell expresses at least nine or more polypeptides encoded by any combinations of the following genes: α-fetal protein (AFP), albumin (ALB), cytokeratin 7 (CK7), cytokeratin 19 (CK19), SOX9, PDX1, PROX1, EpCAM, HHEX and/or HNF4a. In yet another embodiment, the hepatic progenitor cell expresses the following genes (or the polypeptide they encode): α-fetal protein (AFP), albumin (ALB), cytokeratin 7 (CK7), cytokeratin 19 (CK19), SOX9, PDX1, PROX1, EpCAM, HHEX and HNF4a. In some embodiments, the hepatic progenitor cell expresses and can be identified by comparing the level of expression of the following genes or the polypeptides they encode: α-fetal protein (AFP), albumin (ALB), cytokeratin 7 (CK7), cytokeratin 19 (CK19), SOX9, PDX1, PROX1 and/or HNF4a with the level of expression of the same genes/polypeptides in posterior foregut cells. In an embodiment, the hepatic progenitor cells expresses substantially the same amount of albumin than a posterior foregut cell. In an embodiment, the hepatic progenitor cells expresses substantially the same amount of AFP than a posterior foregut cell. In an embodiment, the hepatic progenitor cells expresses more the CK19 gene than a posterior foregut cell. In an embodiment, the hepatic progenitor cells expresses more the CK7 gene than a posterior foregut cell. In an embodiment, the hepatic progenitor cells expresses more the PDX1 gene than a posterior foregut cell. In an embodiment, the hepatic progenitor cells expresses more the SOX9 gene than a posterior foregut cell. In an embodiment, the hepatic progenitor cells expresses more the PROX1 gene than a posterior foregut cell. In an embodiment, the hepatic progenitor cells expresses the HHEX gene, but less than a posterior foregut cell. In an embodiment, the hepatic progentic cells do not substantially express the TRA-1-60 and/or the Nanog genes or express these genes at a very low level when compared to undifferentiated pluripotent cells (such as iPSCs).

The hepatic progenitor cell can be of any origin, it can especially be derived from a mammal and, in some embodiments from a human.

The second culture medium used in the second process can be serum free (e.g., not supplemented with serum). In an alternative embodiment, the second culture medium used in the second process can comprise serum, which can be KnockOut Serum Replacement™ (ThermoFisher Scientific). In an embodiment, the second culture medium comprises between about 0.1 and about 5% (v/v) serum. In still another embodiment, the second culture medium comprises at least about 0.1, 0.2, 0.3, 0.4, 0.5, 0.6, 0.7, 0.8, 0.9, 1, 1.5, 2, 2.5, 3, 3.5, 4, 4.5% or more of serum. In another embodiment, the second culture medium comprises less than about 5, 4.5, 4, 3.5, 3, 2.5, 2, 1.5, 1, 0.9, 0.8, 0.7, 0.6, 0.5, 0.4, 0.3, 0.2% or less of serum. In yet another embodiment, the firs culture medium comprises between about 0.1, 0.2, 0.3, 0.4, 0.5, 0.6, 0.7, 0.8, 0.9, 1, 1.5, 2, 2.5, 3, 3.5, 4 or 4.5% and about 5, 4.5, 4, 3.5, 3, 2.5, 2, 1.5, 1, 0.9, 0.8, 0.7, 0.6, 0.5, 0.4, 0.3 or 0.2% serum. In an embodiment, the second culture medium comprises about 2% serum.

The second culture medium comprises a second set of additives comprising or consisting essentially of an activator of an insulin signaling pathway, an activator of a bone morphogenetic protein (BMP) signaling pathway, an activator of a fibroblast growth factor (FGF) signaling pathway, an activator of an hepatocyte growth factor (HGF) signaling pathway and an activator of a Wnt signaling pathway. As used in the context of the present disclosure, the expression “second culture medium consists essentially of a second set of additives” refers to a second culture medium comprising additional additives which are not essential for the differentiation of the posterior foregut cell into an hepatic progenitor cell but can nevertheless facilitate the differentiation. These additional additives include, but are not limited to, the B27 supplement, retinoic acid, insulin, vitamins and minerals.

The second culture medium also comprises an activator of an insulin signaling pathway. As used in the context of the present disclosure, an “activator of an insulin signaling pathway” refers to a compound capable of activating the signaling pathway associated with the binding of insulin to its cognate insulin receptor (a tyrosine kinase receptor). The compound can either be an agonist of the insulin receptor (insulin, IGF-I or IGF-II), an activator of a polypeptide known to be activated in the insulin signaling pathway and/or an inhibitor of a polypeptide known to be inhibited in the insulin signaling pathway. In an embodiment, the activator is insulin (which can be provided in a recombinant or purified form). In embodiments in which insulin is provided as the activator of the insulin signaling pathway, it can be provided at a concentration of at least about 1, 5, 10, 15, 20, 25, 30, 35, 40, 45, 50, 55, 60, 65, 70, 75, 80, 85, 90, 95 or more ng/mL of the second culture medium. In embodiments in which insulin is provided as the activator of the insulin signaling pathway, it can be provided at a concentration of no more than about 100, 95, 90, 85, 80, 75, 70, 65, 60, 55, 50, 45, 40, 35, 30, 25, 20, 15, 10, 5 or less ng/mL of the second culture medium. In embodiments in which insulin is provided as the activator of the insulin signaling pathway, it can be provided at a concentration of between about 1, 5, 10, 15, 20, 25, 30, 35, 40, 45, 50, 55, 60, 65, 70, 75, 80, 85, 90 or 95 and about 100, 95, 90, 85, 80, 75, 70, 65, 60, 55, 50, 45, 40, 35, 30, 25, 20, 15, 10 or 5 of the second culture medium. In some specific embodiments, insulin can be provided at a concentration of about 10 mg/ml of the second culture medium. In still another embodiment, insulin is provided in the form of the B27 supplement, in the HBM/HCM Bulletkit™ and/or the primary hepatocyte (PHH) supplement.

The second culture medium comprises an activator of a bone morphogenetic protein (BMP) signaling pathway. During development, activators of the BMP signaling pathway are usually being provided by the cardiac mesoderm and favor the differentiation of endodermal cells into posterior foregut cells. As used in the context of the present disclosure, an “activator of a BMP signaling pathway” refers to a compound capable of activating the signaling pathway associated with the binding of a BMP to its cognate receptor (for example BMPR1 and/or BMPR2). Signal transduction the BMP receptors occurs via SMAD and MAP kinase pathways to effect transcription of BMP target genes. The compound can either be an agonist of the BMP receptor (either specific for BMPR1 or BMPR2 or capable of binding and activating both receptors), an activator of a polypeptide known to be activated in the BMP signaling pathway and/or an inhibitor of a polypeptide known to be inhibited in the BMP signaling pathway. Known BMPs include, but are not limited to, BMP1, BMP2, BMP3, BMP4, BMP5, BMP6, BMP7, BMP8a, BMP8b, BMP9, BMP10, BMP11 and BMP15. In an embodiment, the activator is DM3189. In another embodiment, the activator is BMP4 (which can be provided in a recombinant or purified form). BMP4 is a member of the transforming growth factor-β (TGF-β) family binds to two different types of serine-threonine kinase receptors known as BMPR1 and BMPR2. In embodiments in which BMP4 is provided as the activator of the BMP signaling pathway, it can be provided at a concentration of at least about 10, 11, 12, 13, 14, 15, 16, 17, 18, 19, 20, 21, 22, 23, 24, 25, 26, 27, 28, 29 or more ng/mL of the second culture medium. In embodiments in which BMP4 is provided as the activator of the BMP signaling pathway, it can be provided at a concentration of no more than about 30, 29, 28, 27, 26, 25, 24, 23, 22, 21, 20, 19, 18, 17, 16, 15, 14, 13, 12, 11 or less ng/mL of the second culture medium. In embodiments in which BMP4 is provided as the activator of the BMP signaling pathway, it can be provided at a concentration of between about 10, 11, 12, 13, 14, 15, 16, 17, 18, 19, 20, 21, 22, 23, 24, 25, 26, 27, 28 or 29 and about 30, 29, 28, 27, 26, 25, 24, 23, 22, 21, 20, 19, 18, 17, 16, 15, 14, 13, 12 or 11 ng/mL of the second culture medium. In some specific embodiments, BMP4 can be provided at a concentration of about 20 ng/mL of the second culture medium. In additional embodiments, BMP4 can be provided as the activator in both the first and the second set of additives.

The second culture medium also comprises an activator of a fibroblast growth factor (FGF) signaling pathway. During development, activators of the FGF signaling pathway are usually being provided by the cardiac mesoderm and favor the differentiation of endodermal cells into posterior foregut cells. As used in the context of the present disclosure, an “activator of a FGF signaling pathway” refers to a compound capable of activating the signaling pathway associated with the binding of a FGF to its cognate receptor (for example FGFR1, FGFR2, FGFR3 and/or FGFR4). The compound can either be an agonist of the FGF receptor (either specific for FGFR1, FGFR2, FGFR3 and/or FGFR4 or capable of binding and activating more than one receptor), an activator of a polypeptide known to be activated in the FGF signaling pathway and/or an inhibitor of a polypeptide known to be inhibited in the FGF signaling pathway. Known FGFs include, but are not limited to, FGF1, FGF2, FGF3, FGF4, FGF5, FGF6, FGF7, FGF8a, FGF8b, FGF9, FGF10, FGF11, FGF12, FGF13, FGF14, FGF15/19, FGF16, FGF17, FGF18, FGF20, FGF21, FGF22 and FGF23. In an embodiment, the activator is basic FGF or FGF2 (which can be provided in a recombinant or purified form). FGF2 binds to two different types of receptors known as FGFR2 (also known as CD332) and FGFR3. In embodiments in which basic FGF is provided as the activator of the FGF signaling pathway, it can be provided at a concentration of at least about 1, 2, 3, 4, 5, 6, 7, 8, 9, 10, 11, 12, 13, 14, 15, 16, 17, 18, 19 or more ng/mL of the second culture medium. In embodiments in which basic FGF is provided as the activator of the FGF signaling pathway, it can be provided at a concentration of no more than about 20, 19, 18, 17, 16, 15, 14, 13, 12, 11, 10, 9, 8, 7, 6, 5, 4, 3, 2 or less ng/mL of the second culture medium. In embodiments in which basic FGF is provided as the activator of the FGF signaling pathway, it can be provided at a concentration of between about 1, 2, 3, 4, 5, 6, 7, 8, 9, 10, 11, 12, 13, 14, 15, 16, 17, 18 or 19 and about 20, 19, 18, 17, 16, 15, 14, 13, 12, 11, 10, 9, 8, 7, 6, 5, 4, 3, 2 ng/ml of the second culture medium. In some specific embodiments, basic FGF can be provided at a concentration of about 10 ng/mL of the second culture medium. In additional embodiments, basic FGF can be provided as the activator in both the second and the second set of additives.

The second culture medium also comprises an activator of a hepatocyte growth factor (HGF) signaling pathway. During development, activators of the HGF signaling pathway favor the differentiation of endodermal cells into hepatic progenitor cells. As used in the context of the present disclosure, an “activator of a HGF signaling pathway” refers to a compound capable of activating the signaling pathway associated with the binding of HGF to its cognate receptor (for example c-Met). The compound can either be an agonist of the HGF receptor, an activator of a polypeptide known to be activated in the HGF signaling pathway and/or an inhibitor of a polypeptide known to be inhibited in the HGF signaling pathway. In an embodiment, the activator is HGF (which can be provided in a recombinant or purified form). In embodiments in which HGF is provided as the activator of the HGF signaling pathway, it can be provided at a concentration of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or more ng/mL of the second culture medium. In embodiments in which HGF is provided as the activator of the HGF signaling pathway, it can be provided at a concentration of no more than about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or less ng/mL of the second culture medium. In embodiments in which HGF is provided as the activator of the HGF signaling pathway, it can be provided at a concentration of between about 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 and about 40, 39, 38, 37, 36, 35, 34, 33, 32, 31, 30, 29, 28, 27, 26, 25, 24, 23, 22, 21, 20, 19, 18, 17, 16, 15, 14, 13, 12 or 11 ng/mL of the second culture medium. In some specific embodiments, HGF can be provided at a concentration of about 20 ng/mL of the second culture medium.

The second culture medium further comprises an activator of a Wnt signaling pathway. In some embodiments, it is important to activate the Wnt signaling pathway in the posterior foregut cells only after it has previously inhibited (as indicated, for example, in the first process). As used in the context of the present disclosure, an “activator of a Wnt signaling pathway” refers to a compound capable of activating the signaling pathway associated with the binding of a Wnt protein ligand to its cognate Frizzled receptor (for example FZD1, FZD2, FZD3, FZD4, FZD5, FZD6, FZD7, FZD8, FZD9 or FZD10). The family of Frizzled receptors are G protein-coupled receptor proteins. The compound can either be an agonist of the Frizzled receptor (either specific for FZD1, FZD2, FZD3, FZD4, FZD5, FZD6, FZD7, FZD8, FZD9 or FZD10 or capable of binding and activating more than one receptor), an activator of a polypeptide known to be activated in the Wnt signaling pathway and/or an inhibitor of a polypeptide known to be inhibited in the Wnt signaling pathway. Known Wnt proteins include, but are not limited to, WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16. In an embodiment, the activator is Wnt3a, SB-216763 and/or LY2090314. In an embodiment, the activator is capable of inhibiting the biological activity of the GSK3 protein. For example, an activator capable of inhibiting the biological activity of the GSK3 protein can be CHIR99021. In an embodiment in which CHIR99021 is used as the activator of the Wnt signaling pathway, it can be provided at a concentration of at least 0.5, 1, 1.5, 2, 2.5, 3, 3.5, 4, 4.5, 5, 5.5, 6, 6.5, 7, 7.5 μM or more in the second culture medium. In an embodiment in which CHIR99021 is used as the activator of the Wnt signaling pathway, it can be provided at a concentration of no more than 8, 7.5, 7, 6.5, 6, 5.5, 5, 4.5, 4, 3.5, 3, 2.5, 2, 1.5, 1 μM or less in the second culture medium. In an embodiment in which CHIR99021 is used as the activator of the Wnt signaling pathway, it can be provided at a concentration between about 0.5, 1, 1.5, 2, 2.5, 3, 3.5, 4, 4.5, 5, 5.5, 6, 6.5, 7 or 7.5 and about 8, 7.5, 7, 6.5, 6, 5.5, 5, 4.5, 4, 3.5, 3, 2.5, 2, 1.5 or 1 μM in the second culture medium. In an embodiment in which CHIR99021 is used as the inhibitor of the Wnt signaling pathway, it can be provided at a concentration of about 3 μM in the second culture medium.

The second culture medium remains in contact with the posterior foregut cells and the hepatic progenitor cells for at least one day or more to allow differentiation. If the second medium is intended to be in contact with the cultured cells for more than one day, it can be changed daily. In some embodiments of the process of the present disclosure, the second culture medium remains in contact at least 1, 2, 3, 4 or more days with the cultured cells. In another embodiment, the second culture medium remains in contact no more than 5, 4, 3, 2 or less days with the cultured cells. In still another embodiment, the second culture medium remains in contact at least 1, 2, 3, 4 or more days and no more than 5, 4, 3, 2 or less days with the cultured cells. In yet another embodiment, the second culture medium remains in contact between about 1 and 5 days with the cultured cells.

The use of the second culture medium with posterior foregut cells allows the differentiation of posterior foregut cells in hepatic progenitor cells. Therefore, the present disclosure provides a population of hepatic progenitor cells obtained from the process described herein. In the population of hepatic progenitor cells of the present disclosure, the majority of the cells are considered hepatic progenitor cells and can include, in some embodiments, some posterior foregut cells. In an embodiment, the population of hepatic progenitor cells obtained from the second process comprises at least 60, 65, 70, 75, 80, 85, 90, 91, 92, 93, 94, 95, 96, 97, 98 or 99% of hepatic progenitor cells (which can be identified, for example, by determining the expression of CK19 or EpCAM).

The present disclosure provides a third process for making, from an hepatic progenitor cell, an hepatocyte-like cell. The process includes contacting one or more hepatic progenitor cells with a third culture medium comprising a third set of additives (to promote the differentiation of an hepatic progenitor cell into a cell of the hepatocyte lineage), followed by a fourth culture medium comprising a fourth set of additives (to promote the differentiation of the cell of the hepatic lineage into an immature hepatocyte), followed by a fifth culture medium comprising a fifth set of additives (to promote the differentiation of the immature hepatocyte into a mature hepatocyte) under conditions so as to allow the differentiation of the hepatic progenitor cell into an hepatocyte. The hepatic progenitor cells used in the third process can be obtained from performing the first process and/or the second process as described herein.

As used in the present disclosure, an “hepatocyte-like cell” collectively refers to an cell of the hepatocyte lineage, an immature hepatocyte-like cell and a mature hepatocyte-like cell. A cell of the hepatic lineage is not capable of differentiating into a cholangiocyte and is capable of differentiating into an hepatocyte. In some embodiments, hepatocyte-like cells (especially mature hepatocyte-like cells) are cells capable of performing liver-specific functions such as producing specific proteins (albumin, clotting factors, alpha-1-antitrypsin, etc.), detoxifying ammonia into urea, metabolizing drugs, storing glycogen, conjugating bilirubin, synthesizing bile, etc. Hepatocyte-like cells can be identified by those skilled in the art using various techniques known in the art. For example, hepatocyte-like cells can be identified by determining the presence or absence as well as the expression levels of at least one or any combinations of the following genes: α-fetal protein (AFP), albumin (ALB), ASGR1, ASGPR, HNF4a or SOX9 or the polypeptides they encode. In a specific embodiment, the hepatocyte-like cell expresses at least one or any combinations of the following genes: α-fetal protein (AFP), albumin (ALB), ASGR1 (ASGPR), HNF4a and/or SOX9 or the polypeptides they encode. In a specific embodiment, the hepatocyte-like cell expresses at least two or any combinations of the following genes: α-fetal protein (AFP), albumin (ALB), ASGR1 (ASGPR), HNF4a and/or SOX9 or the polypeptides they encode. In a specific embodiment, the hepatocyte-like cell expresses at least three or any combinations of the following genes: α-fetal protein (AFP), albumin (ALB), ASGR1 (ASGPR), HNF4a and/or SOX9 or the polypeptides they encode. In a specific embodiment, the hepatocyte-like cell expresses at least four or any combinations of the following genes: α-fetal protein (AFP), albumin (ALB), ASGR1 (ASGPR), HNF4a and/or SOX9 or the polypeptides they encode. In a specific embodiment, the hepatocyte-like cell expresses the following genes: α-fetal protein (AFP), albumin (ALB), ASGR1 (ASGPR), HNF4a and/or SOX9 or the polypeptides they encode. In still another embodiment, the hepatocyte-like cell can be identified by detecting and optionally measuring the expression of at least one or any combinations of the following genes: α-fetal protein (AFP), albumin (ALB), ASGR1 (ASGPR), HNF4a and/or SOX9 or the polypeptides they encode. In yet another embodiment, the hepatocyte-like cell expresses and can be identified by detecting and optionally measuring the expression of one or more polypeptides encoded by at least one or any combinations of the following genes: α-fetal protein (AFP), albumin (ALB), ASGR1, HNF4a and/or SOX9. In some embodiments, the hepatocyte-like cells expresses and can be identified by comparing the level of expression of the following genes or the polypeptides they encode: α-fetal protein (AFP), albumin (ALB), ASGR1, HNF4a and/or SOX9 with the level of expression of the same genes/polypeptides in an hepatocyte (such as a fetal hepatocyte for example). In specific embodiments, the hepatocyte-like cells expresses more the SOX9 gene or the polypeptides they encode when compared to a corresponding level in a fetal hepatocyte. In specific embodiments, the hepatocyte-like cells express at a substantively same level the HNF4a, AFP, ALB and ASGPR genes, when compared to a fetal hepatocyte. The mature hepatocyte-like cells can have a detectable level of CyP3A4, such as, for example a relative activity of at least 10 000 units per million cells. In still another embodiment, the mature hepatocyte-like cells can have a higher CyP3A4 activity than immature hepatocyte-like cells. The mature hepatocyte-like cells can produce a detectable level of albumin, such as, for example at least about 5, 6, 7, 8, 9, 10, 11, 12 μg/L/10⁶/24 h or more. The mature hepatocyte-like cells can produce a detectable level of albumin, such as, for example at least about 10, 100 or 1 000 μg/L/10⁶/24 h or more.

The hepatocyte-like cell can be of any origin, it can especially be derived from a mammal and, in some embodiments from a human.

The third, fourth and fifth culture medium used in the third process can be serum free (e.g., not supplemented with serum). In an alternative embodiment, the third, fourth and fifth culture medium used in the third process can comprise serum, which can be KnockOut Serum Replacement™ (ThermoFisher Scientific). In an embodiment, the third, fourth and fifth culture medium comprises between about 0.1 and about 5% (v/v) serum. In still another embodiment, the third, fourth and fifth culture medium comprises at least about 0.1, 0.2, 0.3, 0.4, 0.5, 0.6, 0.7, 0.8, 0.9, 1, 1.5, 2, 2.5, 3, 3.5, 4, 4.5% or more of serum. In another embodiment, the third, fourth and fifth culture medium comprises less than about 5, 4.5, 4, 3.5, 3, 2.5, 2, 1.5, 1, 0.9, 0.8, 0.7, 0.6, 0.5, 0.4, 0.3, 0.2% or less of serum. In yet another embodiment, the firs culture medium comprises between about 0.1, 0.2, 0.3, 0.4, 0.5, 0.6, 0.7, 0.8, 0.9, 1, 1.5, 2, 2.5, 3, 3.5, 4 or 4.5% and about 5, 4.5, 4, 3.5, 3, 2.5, 2, 1.5, 1, 0.9, 0.8, 0.7, 0.6, 0.5, 0.4, 0.3 or 0.2% serum. In an embodiment, the third culture medium comprises about 2% serum. In another embodiment, the third culture medium comprises about 1% serum. In another embodiment, the fourth culture medium comprises about 1% serum. In still another embodiment, the fifth culture medium comprises about 1% serum.

The third culture medium comprises a third set of additives comprising or consisting essentially of an activator of an insulin signaling pathway, an activator of a bone morphogenetic protein (BMP) signaling pathway, an activator of a fibroblast growth factor (FGF) signaling pathway, an activator of an hepatocyte growth factor (HGF) signaling pathway, an activator of a Wnt signaling pathway, an inhibitor of the TGFβ signaling pathway, a cytokine and a glucocorticoid. As used in the context of the present disclosure, the expression “third culture medium consists essentially of a third set of additives” refers to a third culture medium comprising additional additives which are not essential for the differentiation of the hepatocyte progenitor cells into hepatocyte-like cells but can nevertheless facilitate the differentiation. These additional additives include, but are not limited to, B27 supplement, primary hepatocyte supplement (PHH), the HBM/HCM Bulletkit™ retinoic acid, insulin, vitamins and minerals.

The third culture medium also comprises an activator of an insulin signaling pathway. As used in the context of the present disclosure, an “activator of an insulin signaling pathway” refers to a compound capable of activating the signaling pathway associated with the binding of insulin to its cognate insulin receptor (a tyrosine kinase receptor). The compound can either be an agonist of the insulin receptor (insulin, IGF-I or IGF-II), an activator of a polypeptide known to be activated in the insulin signaling pathway and/or an inhibitor of a polypeptide known to be inhibited in the insulin signaling pathway. In an embodiment, the activator is insulin (which can be provided in a recombinant or purified form). In embodiments in which insulin is provided as the activator of the insulin signaling pathway, it can be provided at a concentration of at least about 1, 5, 10, 15, 20, 25, 30, 35, 40, 45, 50, 55, 60, 65, 70, 75, 80, 85, 90, 95 or more ng/mL of the third culture medium. In embodiments in which insulin is provided as the activator of the insulin signaling pathway, it can be provided at a concentration of no more than about 100, 95, 90, 85, 80, 75, 70, 65, 60, 55, 50, 45, 40, 35, 30, 25, 20, 15, 10, 5 or less ng/mL of the third culture medium. In embodiments in which insulin is provided as the activator of the insulin signaling pathway, it can be provided at a concentration of between about 1, 5, 10, 15, 20, 25, 30, 35, 40, 45, 50, 55, 60, 65, 70, 75, 80, 85, 90 or 95 and about 100, 95, 90, 85, 80, 75, 70, 65, 60, 55, 50, 45, 40, 35, 30, 25, 20, 15, 10 or 5 of the third culture medium. In some specific embodiments, insulin can be provided at a concentration of about 10 mg/ml of the third culture medium. In still another embodiment, insulin is provided in the form of the B27 supplement, in the HBM/HCM Bulletkit™ and/or the primary hepatocyte (PHH) supplement.

The third culture medium comprises an activator of a bone morphogenetic protein (BMP) signaling pathway. During development, activators of the BMP signaling pathway are usually being provided by the cardiac mesoderm and favor the differentiation of endodermal cells into posterior foregut cells. As used in the context of the present disclosure, an “activator of a BMP signaling pathway” refers to a compound capable of activating the signaling pathway associated with the binding of a BMP to its cognate receptor (for example BMPR1 and/or BMPR2). Signal transduction the BMP receptors occurs via SMAD and MAP kinase pathways to effect transcription of BMP target genes. The compound can either be an agonist of the BMP receptor (either specific for BMPR1 or BMPR2 or capable of binding and activating both receptors), an activator of a polypeptide known to be activated in the BMP signaling pathway and/or an inhibitor of a polypeptide known to be inhibited in the BMP signaling pathway. Known BMPs include, but are not limited to, BMP1, BMP2, BMP3, BMP4, BMP5, BMP6, BMP7, BMP8a, BMP8b, BMP9, BMP10, BMP11 and BMP15. In an embodiment, the activator is DM3189. In another embodiment, the activator is BMP4 (which can be provided in a recombinant or purified form). BMP4 is a member of the transforming growth factor-β (TGF-β) family binds to two different types of serine-threonine kinase receptors known as BMPR1 and BMPR2. In embodiments in which BMP4 is provided as the activator of the BMP signaling pathway, it can be provided at a concentration of at least about 10, 11, 12, 13, 14, 15, 16, 17, 18, 19, 20, 21, 22, 23, 24, 25, 26, 27, 28, 29 or more ng/mL of the third culture medium. In embodiments in which BMP4 is provided as the activator of the BMP signaling pathway, it can be provided at a concentration of no more than about 30, 29, 28, 27, 26, 25, 24, 23, 22, 21, 20, 19, 18, 17, 16, 15, 14, 13, 12, 11 or less ng/mL of the third culture medium. In embodiments in which BMP4 is provided as the activator of the BMP signaling pathway, it can be provided at a concentration of between about 10, 11, 12, 13, 14, 15, 16, 17, 18, 19, 20, 21, 22, 23, 24, 25, 26, 27, 28 or 29 and about 30, 29, 28, 27, 26, 25, 24, 23, 22, 21, 20, 19, 18, 17, 16, 15, 14, 13, 12 or 11 ng/mL of the third culture medium. In some specific embodiments, BMP4 can be provided at a concentration of about 20 ng/mL of the third culture medium. In additional embodiments, BMP4 can be provided as the activator in both the first, the second and the third set of additives.

The third culture medium also comprises an activator of a fibroblast growth factor (FGF) signaling pathway. During development, activators of the FGF signaling pathway are usually being provided by the cardiac mesoderm and favor the differentiation of endodermal cells into posterior foregut cells. As used in the context of the present disclosure, an “activator of a FGF signaling pathway” refers to a compound capable of activating the signaling pathway associated with the binding of a FGF to its cognate receptor (for example FGFR1, FGFR2, FGFR3 and/or FGFR4). The compound can either be an agonist of the FGF receptor (either specific for FGFR1, FGFR2, FGFR3 and/or FGFR4 or capable of binding and activating more than one receptor), an activator of a polypeptide known to be activated in the FGF signaling pathway and/or an inhibitor of a polypeptide known to be inhibited in the FGF signaling pathway. Known FGFs include, but are not limited to, FGF1, FGF2, FGF3, FGF4, FGF5, FGF6, FGF7, FGF8a, FGF8b, FGF9, FGF10, FGF11, FGF12, FGF13, FGF14, FGF15/19, FGF16, FGF17, FGF18, FGF20, FGF21, FGF22 and FGF23. In an embodiment, the activator is basic FGF or FGF2 (which can be provided in a recombinant or purified form). FGF2 binds to two different types of receptors known as FGFR2 (also known as CD332) and FGFR3. In embodiments in which basic FGF is provided as the activator of the FGF signaling pathway, it can be provided at a concentration of at least about 1, 2, 3, 4, 5, 6, 7, 8, 9, 10, 11, 12, 13, 14, 15, 16, 17, 18, 19 or more ng/mL of the first culture medium. In embodiments in which basic FGF is provided as the activator of the FGF signaling pathway, it can be provided at a concentration of no more than about 20, 19, 18, 17, 16, 15, 14, 13, 12, 11, 10, 9, 8, 7, 6, 5, 4, 3, 2 or less ng/mL of the first culture medium. In embodiments in which basic FGF is provided as the activator of the FGF signaling pathway, it can be provided at a concentration of between about 1, 2, 3, 4, 5, 6, 7, 8, 9, 10, 11, 12, 13, 14, 15, 16, 17, 18 or 19 and about 20, 19, 18, 17, 16, 15, 14, 13, 12, 11, 10, 9, 8, 7, 6, 5, 4, 3, 2 ng/ml of the first culture medium. In some specific embodiments, basic FGF can be provided at a concentration of about 10 ng/mL of the third culture medium. In additional embodiments, basic FGF can be provided as the activator in both the second and the third set of additives.

The third culture medium also comprises an activator of a hepatocyte growth factor (HGF) signaling pathway. During development, activators of the HGF signaling pathway favor the differentiation of endodermal cells into cells of the hepatic lineage. As used in the context of the present disclosure, an “activator of a HGF signaling pathway” refers to a compound capable of activating the signaling pathway associated with the binding of HGF to its cognate receptor (for example c-Met). The compound can either be an agonist of the HGF receptor, an activator of a polypeptide known to be activated in the HGF signaling pathway and/or an inhibitor of a polypeptide known to be inhibited in the HGF signaling pathway. In an embodiment, the activator is HGF (which can be provided in a recombinant or purified form). In embodiments in which HGF is provided as the activator of the HGF signaling pathway, it can be provided at a concentration of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or more ng/mL of the third culture medium. In embodiments in which HGF is provided as the activator of the HGF signaling pathway, it can be provided at a concentration of no more than about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or less ng/mL of the third culture medium. In embodiments in which HGF is provided as the activator of the HGF signaling pathway, it can be provided at a concentration of between about 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 and about 40, 39, 38, 37, 36, 35, 34, 33, 32, 31, 30, 29, 28, 27, 26, 25, 24, 23, 22, 21, 20, 19, 18, 17, 16, 15, 14, 13, 12 or 11 ng/mL of the third culture medium. In some specific embodiments, HGF can be provided at a concentration of about 20 ng/mL of the third culture medium. The activator can be HGF in the second and third set of additives.

The third culture medium further comprises an activator of a Wnt signaling pathway. As used in the context of the present disclosure, an “activator of a Wnt signaling pathway” refers to a compound capable of activating the signaling pathway associated with the binding of a Wnt protein ligand to its cognate Frizzled receptor (for example FZD1, FZD2, FZD3, FZD4, FZD5, FZD6, FZD7, FZD8, FZD9 or FZD10). The family of Frizzled receptors are G protein-coupled receptor proteins. The compound can either be an agonist of the Frizzled receptor (either specific for FZD1, FZD2, FZD3, FZD4, FZD5, FZD6, FZD7, FZD8, FZD9 or FZD10 or capable of binding and activating more than one receptor), an activator of a polypeptide known to be activated in the Wnt signaling pathway and/or an inhibitor of a polypeptide known to be inhibited in the Wnt signaling pathway. Known Wnt proteins include, but are not limited to, WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16. In an embodiment, the activator is Wnt3a, SB-216763 and/or LY2090314. In an embodiment, the activator is capable of inhibiting the biological activity of the GSK3 protein. For example, an activator capable of inhibiting the biological activity of the GSK3 protein can be CHIR99021. In an embodiment in which CHIR99021 is used as the activator of the Wnt signaling pathway, it can be provided at a concentration of at least 0.5, 1, 1.5, 2, 2.5, 3, 3.5, 4, 4.5, 5, 5.5, 6, 6.5, 7, 7.5 μM or more in the third culture medium. In an embodiment in which CHIR99021 is used as the activator of the Wnt signaling pathway, it can be provided at a concentration of no more than 8, 7.5, 7, 6.5, 6, 5.5, 5, 4.5, 4, 3.5, 3, 2.5, 2, 1.5, 1 μM or less in the third culture medium. In an embodiment in which CHIR99021 is used as the activator of the Wnt signaling pathway, it can be provided at a concentration between about 0.5, 1, 1.5, 2, 2.5, 3, 3.5, 4, 4.5, 5, 5.5, 6, 6.5, 7 or 7.5 and about 8, 7.5, 7, 6.5, 6, 5.5, 5, 4.5, 4, 3.5, 3, 2.5, 2, 1.5 or 1 μM in the third culture medium. In an embodiment in which CHIR99021 is used as the inhibitor of the Wnt signaling pathway, it can be provided at a concentration of about 3 μM in the third culture medium. In an embodiment, the activator can be CHIR99021 in the second and third set of additives.

The third culture medium further comprises an inhibitor of a transforming growth factor β (TGFβ) signaling pathway. The presence of the inhibitor of the TGFβ signaling pathway, in combination with the presence of an inhibitor of the Wnt signaling pathway, favors the expression of the HEX and PROX1 genes which encode polypeptides required for liver development. As used in the context of the present disclosure, an “inhibitor of a TGFβ signaling pathway” refers to a compound capable of inhibiting the signaling pathway associated with the binding of TGFβ to its cognate receptor. The family of TGFβ receptors mediate signalization via the SMAD proteins. The compound can either be an antagonist of the TGFβ receptor, an inhibitor of a polypeptide known to be activated in the TGFβ signaling pathway and/or an activator of a polypeptide known to be inhibited in the TGFβ signaling pathway. Known TGFβ proteins include, but are not limited to, TGFB1, TGFB2, TGFB3 and TGFB4. In an embodiment, the inhibitor is capable of inhibiting the biological activity of at least one of the ALK4, ALK5 or ALK7 polypeptides. In some embodiments, the inhibitor is capable of inhibiting the biological activity of the ALK4, ALK5 and ALK7 polypeptides. For example, the inhibitor capable of inhibiting the biological activity of the ALK4, ALK5 and ALK7 polypeptides can be A83-01. Alternatively or in combination, the inhibitor can be SB431542 and/or LY364947. In an embodiment in which A83-01 is used as the inhibitor of the TGFβ signaling pathway, it can be provided at a concentration of at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5 μM or more in the third culture medium. In an embodiment in which A83-01 is used as the inhibitor of the TGFβ signaling pathway, it can be provided at a concentration of no more than 5, 4.5, 4., 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 μM or less in the third culture medium. In an embodiment in which A83-01 is used as the inhibitor of the TGFβ signaling pathway, it can be provided at a concentration between 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4 or 4.5 and about 5, 4.5, 4., 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3 or 0.2 μM in the third culture medium. In an embodiment in which A83-01 is used as the inhibitor of the TGFβ signaling pathway, it can be provided at a concentration of about 1 μM in the second culture medium. In some embodiments, A83-01 can be the inhibitor in the first and third set of additives.

The third medium comprises also comprises a cytokine, such as, for example oncostatin M (OSM). In embodiments in which oncostatin M is used as the cytokine, it can be present at a concentration of at least 10, 11, 12, 13, 14, 15, 16, 17, 18, 19, 20, 21, 22, 23, 24, 25, 26, 27, 28, 29 ng/ml or higher in the third culture medium. In embodiments in which oncostatin M is used as the cytokine, it can be present at a concentration of no more than 30, 29, 28, 27, 26, 25, 24, 23, 22, 21, 20, 19, 18, 17, 16, 15, 14, 13, 12, 11 ng/ml or lower in the third culture medium. In embodiments in which oncostatin M is used as the cytokine, it can be present at a concentration between about 10, 11, 12, 13, 14, 15, 16, 17, 18, 19, 20, 21, 22, 23, 24, 25, 26, 27, 28 or 29 and about 30, 29, 28, 27, 26, 25, 24, 23, 22, 21, 20, 19, 18, 17, 16, 15, 14, 13, 12 or 11 ng/ml in the third culture medium. In a specific embodiment, oncostatin M is present at a concentration of about 20 ng/ml in the third culture medium.

The third medium further comprises a glucocorticoid, such as, for example, dexamethasone. In embodiments in which dexamethasone is used as the glucocorticoid, it can be present at a concentration of at least 5, 6, 7, 8, 9, 10, 11, 12, 13, 14 μM or higher in the third culture medium. In embodiments in which dexamethasone is used as the glucocorticoid, it can be present at a concentration of no more than 15, 14, 13, 12, 11, 10, 9, 8, 7, 6 μM or lower in the third culture medium. In embodiments in which dexamethasone is used as the glucocorticoid, it can be present at a concentration between about 5, 6, 7, 8, 9, 10, 11, 12, 13 or 14 and about 15, 14, 13, 12, 11, 10, 9, 8, 7 or 6 μM in the third culture medium. In a specific embodiment, dexamethasone is present at a concentration of about 10 μM in the third culture medium.

The third culture medium remains in contact with the hepatic progenitor cells and the cells of the hepatocyte lineage for at least one day or more to allow differentiation. If the third medium is intended to be in contact with the cultured cells for more than one day, it can be changed daily. In some embodiments of the process of the present disclosure, the third culture medium remains in contact at least 1, 2, 3, 4 or more days with the cultured cells. In another embodiment, the third culture medium remains in contact no more than 5, 4, 3, 2 or less days with the cultured cells. In still another embodiment, the third culture medium remains in contact at least 1, 2, 3, 4 or more days and no more than 5, 4, 3, 2 or less days with the cultured cells. In yet another embodiment, the third culture medium remains in contact between about 1 and 5 days with the cultured cells.

The use of the third culture medium with posterior foregut cells allows the differentiation of hepatic progenitor cells into cells of the hepatocyte lineage. Therefore, the present disclosure provides a population of cells of the hepatocyte lineage obtained from the process described herein. In the population of cells of the hepatocyte lineage of the present disclosure, the majority of the cells are considered cells of the hepatocyte lineage and can include, in some embodiments, some hepatic progenitor cells and/or endodermal cells.

The fourth culture medium comprises a fourth set of additives comprising or consisting essentially of an activator of the insulin signaling pathway, a cytokine and a glucocorticoid. As used in the context of the present disclosure, the expression “fourth culture medium consists essentially of a fourth set of additives” refers to a fourth culture medium comprising additional additives which are not essential for the differentiation of the cells of the hepatocyte lineage into immature hepatocyte-like cells but can nevertheless facilitate the differentiation. These additional additives include, but are not limited to, B27 supplement, primary hepatocyte supplement (PHH), insulin, the HBM/HCM Bulletkit™ retinoic acid, vitamins and minerals.

The fourth culture medium also comprises an activator of an insulin signaling pathway. As used in the context of the present disclosure, an “activator of an insulin signaling pathway” refers to a compound capable of activating the signaling pathway associated with the binding of insulin to its cognate insulin receptor (a tyrosine kinase receptor). The compound can either be an agonist of the insulin receptor (insulin, IGF-I or IGF-II), an activator of a polypeptide known to be activated in the insulin signaling pathway and/or an inhibitor of a polypeptide known to be inhibited in the insulin signaling pathway. In an embodiment, the activator is insulin (which can be provided in a recombinant or purified form). In embodiments in which insulin is provided as the activator of the insulin signaling pathway, it can be provided at a concentration of at least about 1, 5, 10, 15, 20, 25, 30, 35, 40, 45, 50, 55, 60, 65, 70, 75, 80, 85, 90, 95 or more ng/mL of the fourth culture medium. In embodiments in which insulin is provided as the activator of the insulin signaling pathway, it can be provided at a concentration of no more than about 100, 95, 90, 85, 80, 75, 70, 65, 60, 55, 50, 45, 40, 35, 30, 25, 20, 15, 10, 5 or less ng/mL of the fourth culture medium. In embodiments in which insulin is provided as the activator of the insulin signaling pathway, it can be provided at a concentration of between about 1, 5, 10, 15, 20, 25, 30, 35, 40, 45, 50, 55, 60, 65, 70, 75, 80, 85, 90 or 95 and about 100, 95, 90, 85, 80, 75, 70, 65, 60, 55, 50, 45, 40, 35, 30, 25, 20, 15, 10 or 5 of the fourth culture medium. In some specific embodiments, insulin can be provided at a concentration of about 10 mg/ml of the fourth culture medium. In still another embodiment, insulin is provided in the form of the B27 supplement, the HBM/HCM Bulletkit™ and/or the primary hepatocyte (PHH) supplement.

The fourth medium comprises also comprises a cytokine, such as, for example oncostatin M (OSM). In embodiments in which oncostatin M is used as the cytokine, it can be present at a concentration of at least 10, 11, 12, 13, 14, 15, 16, 17, 18, 19, 20, 21, 22, 23, 24, 25, 26, 27, 28, 29 ng/ml or higher in the fourth culture medium. In embodiments in which oncostatin M is used as the cytokine, it can be present at a concentration of no more than 30, 29, 28, 27, 26, 25, 24, 23, 22, 21, 20, 19, 18, 17, 16, 15, 14, 13, 12, 11 ng/ml or lower in the fourth culture medium. In embodiments in which oncostatin M is used as the cytokine, it can be present at a concentration between about 10, 11, 12, 13, 14, 15, 16, 17, 18, 19, 20, 21, 22, 23, 24, 25, 26, 27, 28 or 29 and about 30, 29, 28, 27, 26, 25, 24, 23, 22, 21, 20, 19, 18, 17, 16, 15, 14, 13, 12 or 11 ng/ml in the fourth culture medium. In a specific embodiment, oncostatin M is present at a concentration of about 20 ng/ml in the fourth culture medium.

The fourth medium further comprises a glucocorticoid, such as, for example, dexamethasone. In embodiments in which dexamethasone is used as the glucocorticoid, it can be present at a concentration of at least 5, 6, 7, 8, 9, 10, 11, 12, 13, 14 μM or higher in the fourth culture medium. In embodiments in which dexamethasone is used as the glucocorticoid, it can be present at a concentration of no more than 15, 14, 13, 12, 11, 10, 9, 8, 7, 6 μM or lower in the fourth culture medium. In embodiments in which dexamethasone is used as the glucocorticoid, it can be present at a concentration between about 5, 6, 7, 8, 9, 10, 11, 12, 13 or 14 and about 15, 14, 13, 12, 11, 10, 9, 8, 7 or 6 μM in the fourth culture medium. In a specific embodiment, dexamethasone is present at a concentration of about 10 μM in the fourth culture medium.

The fourth culture medium remains in contact with the cells of the hepatocyte lineage and the immature hepatocyte-like cells for at least one day or more to allow differentiation. If the fourth medium is intended to be in contact with the cultured cells for more than one day, it can be changed daily. In some embodiments of the process of the present disclosure, the fourth culture medium remains in contact at least 1, 2, 3, 4 or more days with the cultured cells. In another embodiment, the fourth culture medium remains in contact no more than 5, 4, 3, 2 or less days with the cultured cells. In still another embodiment, the fourth culture medium remains in contact at least 1, 2, 3, 4 or more days and no more than 5, 4, 3, 2 or less days with the cultured cells. In yet another embodiment, the fourth culture medium remains in contact between about 1 and 5 days with the cultured cells.

The use of the fourth culture medium with posterior foregut cells allows the differentiation of cells of the hepatocyte lineage into immature hepatocyte-like cells. Therefore, the present disclosure provides a population of immature hepatocyte-like cells obtained from the process described herein. In the population of immature hepatocyte-like cells of the present disclosure, the majority of the cells are considered to be immature hepatocyte-like cells and can include, in some embodiments, some cells of the hepatocyte lineage, hepatic progenitor cells and/or endodermal cells.

The fifth culture medium comprises a fifth set of additives comprising or consisting essentially of an activator of the insulin signaling pathway and a glucocorticoid. The fifth culture medium and the fifth set of additives exclude cytokines, such as, for example, oncostatin M. As used in the context of the present disclosure, the expression “fifth culture medium consists essentially of a fifth set of additives” refers to a fifth culture medium comprising additional additives which are not essential for the differentiation of immature hepatocyte-like cells in mature hepatocyte-like cells but can nevertheless facilitate the differentiation. These additional additives include, but are not limited to, B27 supplement, primary hepatocyte supplement, retinoic acid, insulin, vitamins, the HBM/HCM Bulletkit™ and minerals.

The fifth culture medium also comprises an activator of an insulin signaling pathway. As used in the context of the present disclosure, an “activator of an insulin signaling pathway” refers to a compound capable of activating the signaling pathway associated with the binding of insulin to its cognate insulin receptor (a tyrosine kinase receptor). The compound can either be an agonist of the insulin receptor (insulin, IGF-I or IGF-II), an activator of a polypeptide known to be activated in the insulin signaling pathway and/or an inhibitor of a polypeptide known to be inhibited in the insulin signaling pathway. In an embodiment, the activator is insulin (which can be provided in a recombinant or purified form). In embodiments in which insulin is provided as the activator of the insulin signaling pathway, it can be provided at a concentration of at least about 1, 5, 10, 15, 20, 25, 30, 35, 40, 45, 50, 55, 60, 65, 70, 75, 80, 85, 90, 95 or more ng/mL of the fifth culture medium. In embodiments in which insulin is provided as the activator of the insulin signaling pathway, it can be provided at a concentration of no more than about 100, 95, 90, 85, 80, 75, 70, 65, 60, 55, 50, 45, 40, 35, 30, 25, 20, 15, 10, 5 or less ng/mL of the fifth culture medium. In embodiments in which insulin is provided as the activator of the insulin signaling pathway, it can be provided at a concentration of between about 1, 5, 10, 15, 20, 25, 30, 35, 40, 45, 50, 55, 60, 65, 70, 75, 80, 85, 90 or 95 and about 100, 95, 90, 85, 80, 75, 70, 65, 60, 55, 50, 45, 40, 35, 30, 25, 20, 15, 10 or 5 of the fifth culture medium. In some specific embodiments, insulin can be provided at a concentration of about 10 mg/ml of the fifth culture medium. In still another embodiment, insulin is provided in the form of the B27 supplement, in the HBM/HCM Bulletkit™ and/or the primary hepatocyte (PHH) supplement.

The fifth medium further comprises a glucocorticoid, such as, for example, dexamethasone. In embodiments in which dexamethasone is used as the glucocorticoid, it can be present at a concentration of at least 5, 6, 7, 8, 9, 10, 11, 12, 13, 14 μM or higher in the fifth culture medium. In embodiments in which dexamethasone is used as the glucocorticoid, it can be present at a concentration of no more than 15, 14, 13, 12, 11, 10, 9, 8, 7, 6 μM or lower in the fifth culture medium. In embodiments in which dexamethasone is used as the glucocorticoid, it can be present at a concentration between about 5, 6, 7, 8, 9, 10, 11, 12, 13 or 14 and about 15, 14, 13, 12, 11, 10, 9, 8, 7 or 6 μM in the fifth culture medium. In a specific embodiment, dexamethasone is present at a concentration of about 10 μM in the fifth culture medium. In a specific embodiment, dexamethasone is present at a concentration of about 10 μM in the fifth culture medium.

The fifth culture medium remains in contact with the immature and mature hepatocyte-like cells for at least one day or more to allow differentiation. If the fifth medium is intended to be in contact with the cultured cells for more than one day, it can be changed daily. In some embodiments of the process of the present disclosure, the fifth culture medium remains in contact at least 1, 2, 3, 4 or more days with the cultured cells. In another embodiment, the fifth culture medium remains in contact no more than 5, 4, 3, 2 or less days with the cultured cells. In still another embodiment, the fifth culture medium remains in contact at least 1, 2, 3, 4 or more days and no more than 5, 4, 3, 2 or less days with the cultured cells. In yet another embodiment, the fifth culture medium remains in contact between about 1 and 5 days with the cultured cells.

The use of the fifth culture medium with posterior foregut cells allows the differentiation of immature hepatocyte-like cells into mature hepatocyte-like cells. Therefore, the present disclosure provides a population of mature hepatocyte-like cells obtained from the process described herein. In the population of mature hepatocyte-like cells of the present disclosure, the majority of the cells are considered to be mature hepatocyte-like cells and can include, in some embodiments, some immature hepatocyte-like cells, cells of the hepatic lineage, hepatic progenitor cells and/or endodermal cells.

The culture medium described herein specifically exclude having EGF, as it can promote formation of the biliary cells.

The present disclosure provides combining the first, second and/or third process as disclosed herein. For example, the first process can be combined with the second process to make hepatic progenitor cells from endodermal cells. In another example, the second process can be combined with the third process to make hepatocyte-like cells from posterior foregut cells. In a further example, the first, second and third processes can be combined to make hepatocyte-like cells from endodermal cells. The processes described herein generate high number of hepatocyte-like cells and/or hepatocyte-like cells having more potent biological activity (e.g., higher Cyp3A4 activity, higher albumin expression levels and/or higher urea production levels) and/or capable of metabolizing therapeutic agents (or potential therapeutic agents). This specific embodiment is especially useful for making hepatocyte-like cells intended to be included in an encapsulated liver tissue as indicated below since it provides a

The present disclosure also provides components for kits making posterior foregut cells, hepatic progenitor cells and/or hepatocyte-like cells. Broadly, the kit comprises at least one set of additives as described herein or at least one culture medium as described herein, optionally a cell, as well as instructions to conduct the processes described herein. Kits for making posterior foregut cells can include, for example, a first set of additives or a first culture medium, optionally endodermal cells as well as instructions for conducting the first process. Kits for making hepatic progenitor cells can include, for example, a second set of additives or a second culture medium, optionally posterior foregut cells as well as instructions for conducting the second process. Kits for making hepatocyte-like cells can include, for example, a third set of additives or a third culture medium, a fourth set of additives or a fourth culture medium, a fifth set of additives or a fifth culture medium, optionally hepatic progenitor cells, cells of the hepatic lineage or immature hepatocyte-like cells as well as instructions for conducting the third process.

Encapsulated Liver Tissue

The encapsulated liver tissue comprises at least one (and in an embodiment a plurality of) liver organoid that is at least partially covered with a biocompatible cross-linked polymer. As used in the context of the present disclosure, a “liver organoid” refers to a mixture of cultured hepatic, mesenchymal and, optionally endothelial cells, in which the hepatic cells have been obtained using the process described herein. In some embodiments, the liver organoid comprises a mixture of cultured hepatic, mesenchymal and endothelial cells. The liver organoid is generally spherical in shape and its surface may be irregular. The relative diameter of the liver organoid is between about 50 and about 500 μm. The cellular core of the liver is composed of hepatic cells, mesenchymal cells and, optionally, endothelial cells and, in some embodiments, the extracellular matrix, the hepatic, mesenchymal and, optionally the endothelial cells have produced and assembled while being cultured. The liver organoid can be obtained by culturing the cells in suspension. In some embodiments, particularly prior to the culture/differentiation of the encapsulated liver tissue, the surface of the liver organoid is at least partially covered (and in some embodiments substantially covered) with hepatic cells, such as, for example, hepatocytes and/or biliary epithelial cells. In another embodiment, the hepatic cells are dispersed throughout (but not necessary homogeneously) the cellular core. The organoids present in the encapsulated liver tissue are at least partially covered (and in some embodiments substantially covered) with a first biocompatible cross-linked polymer.

Prior to being encapsulated, the liver organoid is free of exogenous extracellular matrix. The liver organoid is substantially composed of the cultured hepatic, mesenchymal and, optionally, endothelial cells. Furthermore, the liver organoid (encapsulated or not in the first biocompatible polymer) exhibits liver functions, for example, the liver organoid is capable of synthesizing albumin as well clotting factors, exhibiting CyP3A4 activity, detoxifying ammonia to urea and performing liver-specific metabolism of drugs (i.e. tacrolimus or rifampicin).

The liver organoids of the present disclosure are substantially spherical in shape and have a relative diameter in the micrometer range (e.g., it is smaller than 1 mm in diameter). In an embodiment, the liver organoid, prior to its encapsulation, has a relative diameter of at least about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480 or 490 μm. In yet another embodiment, the liver organoid, prior to its encapsulation, has a relative diameter equal to or lower than about 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70 or 60 μm. In another embodiment, the liver organoid, prior to its encapsulation, has a relative diameter between at least about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480 or 490 μm and equal to or lower than about 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70 or 60 pm. In some embodiments, the liver organoid, prior to its encapsulation, has a relative diameter between at least about 100, 110, 120, 130, 140, 150, 160, 170, 180, 190, 200, 210, 220, 230, 240, 250, 260, 270, 280 or 290 μm about and equal to or lower than about 300, 290, 280, 270, 260, 250, 240, 230, 220, 210, 200, 190, 180, 170, 160, 150, 140, 130, 120, 110, 100, 90, 80, 70 or 60 μm. In still yet another embodiment, the liver organoid prior to its encapsulation, has a relative diameter of at least about 100 μm and equal to or lower than about 300 μm. For example, the liver organoid, prior to its encapsulation, has a relative diameter of at least about 150, 160, 170, 180 or 190 μm and lower than 200, 190, 180, 170 or 160 μm. In yet a further embodiment, the liver organoid, prior to its encapsulation, has a relative diameter of at least about 150 μm and equal to lower than about 200 μm. The size of the liver organoids allows the cells it contains to increase their exposure to various nutrients and to biological fluid/cells in contact with the encapsulated liver tissue. In some embodiments, this allows the liver organoids to be able to remain viable and biologically active in vivo without the need to vascularize them with the host's vascular system (e.g., the vascular system of the host having received the encapsulated liver tissue).

The hepatic cells of the liver organoid can be dispersed through the entire organoid, and, in some embodiments, some of them can be located at the surface of the cellular core of the liver organoid. The hepatic cells of the liver organoid can be, for example, cells from the definitive endoderm, posterior foregut cells, cells of the hepatocyte lineage or hepatic progenitor cells or hepatocyte-like cells. The hepatic cells of the liver organoid can be hepatocyte-like cells and/or biliary epithelial cells. The hepatic cells of the liver organoid can be from a single cell type (e.g., definitive endoderm cells, posterior foregut cells, cells of the hepatocyte lineage, hepatocyte-like cells or biliary epithelial cells) or from a mixture of cell types (e.g., a mixture of at least two of the following cell types: definitive endoderm cells, posterior foregut cells, cells of the hepatocyte lineage, hepatocyte-like cells and/or biliary epithelial cells). During the in vitro cell culture of the liver organoid or even when the liver organoid is placed in vivo, the phenotype of the hepatic cell type(s) can change or the hepatic cell can differentiate. For example, the hepatic cells of the liver organoid can differentiate (from definitive endoderm, posterior foregut or cells of the hepatocyte lineage to hepatocyte-like cells or biliary epithelial cells) during co-culture with mesenchymal and optionally endothelial cells or when placed in vivo. In order to determine if hepatocyte-like cells are present in the liver organoids, the activity of cytochrome P450 family 3 subfamily A member 4 (CyP3A4) can be determined by means known in the art. The synthesis/production of albumin, clotting factors and urea, as well as the activity of CyP3A4, can also be monitored to determine if hepatocyte-like cells are present in the liver organoid. In order to determine if definitive endoderm or posterior foregut cells are present in the liver organoids, the expression of SOX17, FOXA2, CXCR4, GATA4 can be determined by means known in the art.

The mesenchymal cells of the liver organoid can be, for example, mesenchymal stem/progenitor cells of different origins (bone marrow (including blood), umbilical cord or adipose tissue), adipocytes, muscle cells, hepatic stellate cells, myofibroblasts and/or fibroblasts. The mesenchymal cells of the liver organoid can be from a single cell type (e.g., mesenchymal stem/progenitor cells, adipocyte, muscle cells or fibroblasts) or from a mixture of cell types (e.g., a mixture of at least two of the following cell types: mesenchymal stem/progenitor cells, adipocyte, muscle cells, hepatic stellate cells, myofibroblasts and/or fibroblasts). The type of mesenchymal cells of the liver organoid can differentiate (from mesenchymal stem/progenitor cells to fibroblasts, adipocytes or muscle cells) during co-culture with hepatic and optionally endothelial cells or when placed in vivo. Mesenchymal stem/progenitor cells are known to express, amongst others genes, a smooth-muscle actin (αSMA), fibronectin, CD90 and CD73. In order to determine the location or presence of mesenchymal cells in a liver organoid, it is possible, amongst other things, to determine the expression of genes or proteins specific or associated to the mesenchymal lineage.

The endothelial cells of the liver organoid, when present, can be, for example, endothelial progenitor cells and/or endothelial cells of various origins. The endothelial cells of the liver organoid can be from a single cell type (e.g., endothelial progenitor cells or endothelial cells) or from a mixture of cell types (e.g., a mixture of endothelial progenitor cells and endothelial cells). The type endothelial cells of the liver organoid can differentiate (from endothelial progenitor cells to endothelial cells) during in vitro co-culture with endodermal and mesenchymal cells or when placed in vivo. In some embodiments, the endothelial cells of the liver organoid can organize in a capillary or a capillary-like configuration in which endothelial cells line up the internal surface of a lumen (which can be partial).

As indicated above, the cellular core of the liver organoid is composed of hepatic, mesenchymal and optionally endothelial cells and, in some embodiments, of a extracellular matrix produced and assumed by the cells during culture. The cellular core of the liver organoid is substantially poor in necrotic/apoptotic cells (e.g., it does not have necrotic areas when examined by histology) because nutrients from the medium in which the liver organoids are cultured can diffuse across the cellular core and thus can be delivered to cells within the cellular core and the metabolic waste products of the cells of the cellular core can diffuse out of the liver organoid. The liver organoid itself (prior to encapsulation) does not include (e.g., is free from) exogenous extracellular matrix or synthetic polymeric material. In some embodiments, the hepatic cells can be present on the surface of the cellular core. In another embodiment, the hepatic cells can, in combination with the cells of the cellular core, produce and assemble extracellular matrix material (collagen and fibronectin for example) and, in some embodiment, basal membrane material.

As indicated above, the hepatic cells can cover at least partially the surface of the cellular core of the liver organoid. In the context of the present disclosure, the expression “hepatic cells cover at least partially the surface of the cellular core” indicate that the hepatic cells occupy at least about 10%, 20%, 30% or 40% of the surface of the cellular core. In some embodiments, the hepatic cells substantially cover the surface of the cellular core. In the context of the present disclosure, the expression “hepatic cells substantially cover the surface of the cellular core” indicate that the hepatic cells occupy the majority of the surface of the cellular core, for example, at least about 50%, 60%, 70%, 80%, 90%, 95%, 99% of the surface of the cellular core. In an embodiment, the hepatic cells completely cover the surface of the cellular core (e.g., more than 99% of the surface of the cellular core is covered with hepatic cells).

In an embodiment, the liver organoids of the present disclosure, before encapsulation in the first cross-linked biocompatible polymer, have a higher proportion of mesenchymal (and when present endothelial) cells than hepatocyte-like cells and/or biliary epithelial cells than what is observed in the mammalian liver. However, after encapsulation in the first cross-linked biocompatible polymer, the liver organoids of the present disclosure have a higher proportion of hepatic cells when compared to mesenchymal (and when present endothelial) cells. It is known that the mammalian liver is composed of about 90% hepatic cells. As such, in some embodiments of the present disclosure, the proportion of hepatic cells in the liver organoids is lower than about 90%, 85%, 80% or 75% (in comparison to the total number of cells of the liver organoid).

Liver organoids can be made from cells of different origin. In an embodiment, at least one of the hepatic, mesenchymal or endothelial cells are from a mammal, for example a human. In another embodiment, at least two of the hepatic, mesenchymal or endothelial cells are from a mammal, for example a human. In still another embodiment, the hepatic, mesenchymal and endothelial cells are all from a mammal, for example a human. Within the liver organoid, cells from different origin can be combined. For example, the mesenchymal and endothelial cells can be from murine or porcine origin while the hepatic cells can be from human origin. These combinations are not exhaustive and the person skilled in the art will envisage additional combinations that can be suitable in the context of the present disclosure.

The cells of the liver organoid can be derived from different sources. For example, the cells of the liver organoid can be derived from a primary cell culture, an established cell line or a differentiated stem cell. Within the liver organoid, cells from different sources can be combined. For example, the hepatic cells can be from a primary cell culture, the mesenchymal cells can be from an established cell line and the endothelial cell can be from a differentiated cell line. Alternatively, within the liver organoid, cells from the same source (for example differentiated stem cells) can also be combined. This embodiment is especially useful since it allows obtaining the cells for making encapsulated liver tissue from a single cellular source (e.g., a stem cell). In a specific embodiment, the cells of the liver organoid are derived from a single stem cell population which has been differentiated in hepatic, mesenchymal and, optionally, endothelial cells. The stem cell population can be from an embryonic stem cell or an induced pluripotent stem cell. In a specific embodiment, the cells of the liver organoid are derived from a single pluripotent stem cell population which has been differentiated in hepatic, mesenchymal and, optionally, endothelial cells.

The polymer (also referred to as a polymeric matrix) that can be used in the encapsulated liver tissue forms an hydrogel around the liver organoid(s). As known in the art, an hydrogel refers to polymeric chains that are hydrophilic in which water is the dispersion medium. Hydrogels can be obtained from natural or synthetic polymeric networks. In the context of the present disclosure, encapsulation within the hydrogel prevents embedded liver organoids from leaking out of the polymer, thus eliminating or reducing the risk that cells of the liver organoids could give rise to an immune reaction or a tumor within the recipient's body upon implantation. In an embodiment, each liver organoid is encapsulated individually and the encapsulated liver organoids can, in another embodiment, be further included in a polymeric matrix. In still another embodiment, the liver organoids are included in a polymeric matrix so as to encapsulate them.

In the context of the present disclosure, a polymer is considered “biocompatible” when is it does not exhibit toxicity when introduced into a subject (e.g., a human for example). In the context of the present disclosure, it is preferable that the biocompatible polymer does not exhibit toxicity towards the cells of the liver organoid or when placed in vivo in a subject (e.g., a human for example). Hepatotoxicity can be measured, for example, by determining hepatocyte-like cells apoptotic death rate (e.g., wherein an increase in apoptosis is indicative of hepatotoxicity), transaminase levels (e.g., wherein an increase in transaminase levels is indicative of hepatotoxicity), ballooning of the hepatocyte-like cells (e.g., wherein an increase in ballooning is indicative of hepatotoxicity), microvesicular steatosis in the hepatocyte-like cells (e.g., wherein an increase in steatosis is indicative of hepatotoxicity), biliary cells death rate (e.g., wherein an increase in biliary cells death rate is indicative of hepatotoxicity), γ-glutamyl transpeptidase (GGT) levels (e.g., wherein an increase in GGT levels is indicative of hepatotoxicity). Biocompatible polymers include, but are not limited to, carbohydrates (glycosaminoglycan such as hyaluronic acid (HA), chondroitin sulphate, dermatan sulphate, keratan sulphate, heparan sulphate, alginate, chitosan, heparin, agarose, dextran, cellulose, and/or derivatives thereof), proteins (collagen, elastin, fibrin, albumin, poly (amino acid), glycoprotein, antibody and/or derivatives thereof) and/or synthetic polymers (e.g., based on poly(ethylene glycol) (PEG), poly(hydroxyethyl methacrylate) (PHEMA) and/or poly(vinyl alcohol) (PVA)). The biocompatible polymer can be a single polymer or a mixture of different polymers (for example those described in US2012/0142069). Exemplary biocompatible polymers includes, but are not limited to, poly(ethylene) glycol, polylactic acid (PLA), polyglycolic acid (PGA), polycaprolactone (PCL), fibrin, polysaccharidic materials (like chitosan, proteoglycans or glycosaminoglycans (GAGs)), alginate, collagen, thiolated heparin and mixtures thereof. In some embodiments, the biocompatible polymers can be linear, branched and optionally grafted with peptides (e.g., RGD), growth factors, integrins or drugs.

In some embodiments, the polymer is “low-immunogenic polymer” and does not elicit or elicits only a minimal (i.e. not resulting in a degradation, modification or loss of function of the polymer) immune response in the recipient. This low-immunogenic polymer is also capable of masking one or more antigenic determinant of a cell and lowering or even preventing an immune response to the antigenic determinant when such an antigenic determinant is introduced into an allogeneic subject.

The polymer present in the encapsulated liver tissue of the present disclosure are preferably cross-linkable, e.g., capable of being cross-linked. The polymers can be cross-linked thermally, chemically (e.g., by using one or more peptides, such as, VPMS, RGD, etc.) or by the use of pH or light (e.g., photopolymerization, using UV light for example). In some embodiments, cross-linking can be carried out after the liver organoids (encapsulated or not by a polymeric matrix) have been dispersed within the polymeric matrix.

The polymers of the present disclosure can either be totally or partially biodegradable (e.g., susceptible of being hydrolyzed by the metabolism of a living organism) or totally or partially resistant to biodegradation (e.g., resistant to hydrolysis when subjected to the metabolism of a living organism). Exemplary biocompatible and biodegradable polymers include, but are not limited to poly(ethylene-glycol)-maelimide (PEG-Mal) 8-arm. Exemplary biocompatible and biodegradation-resistant polymers include, but are not limited to, poly(ethylene-glycol)-vinyl sulfone (PEG-VS).

The encapsulated liver tissue comprises a first biocompatible and cross-linked polymer which at least partially (and in some instances substantially) covers the liver organoid. The first biocompatible polymer is in physical contact with the cells of the liver organoids. In the context of the present disclosure, the expression “liver organoid(s) at least partially covered by the first biocompatible and cross-linked polymer” indicates that the first biocompatible and cross-linked polymer occupies at least about 10%, 20%, 30% or 40% of the surface of the liver organoid. In some embodiments, the first biocompatible and cross-linked polymer substantially covers the surface of the liver organoid(s). In the context of the present disclosure, the expression “liver organoid(s) substantially covered by the first biocompatible and cross-linked polymer” indicates that the first biocompatible and cross-linked polymer occupies the majority of the surface of the liver organoid, for example, at least about 50%, 60%, 70%, 80%, 90%, 95%, 99% of the surface of the organoid. In an embodiment, the first biocompatible and cross-linked polymer completely covers the surface of the liver organoid (e.g., more than 99% of the surface of the liver organoid is covered with the first biocompatible and cross-linked polymer).

In some embodiments, the encapsulated liver tissue can also comprise a second biocompatible and cross-linked polymer which at least partially (and in some instances substantially) covers the first biocompatible and cross-linked polymer. The second biocompatible polymer is in physical contact with the first biocompatible cross-linked and, in embodiments, with the cells of the liver organoid. In the context of the present disclosure, the expression “first biocompatible cross-linked polymer at least partially covered by the second biocompatible and cross-linked polymer” indicates that the second biocompatible and cross-linked polymer occupies at least about 10%, 20%, 30% or 40% of the surface of the first biocompatible and cross-linked first polymer. In some embodiments, the second biocompatible and cross-linked polymer substantially covers the surface of the first biocompatible and cross-linked polymer. In the context of the present disclosure, the expression “first biocompatible and cross-linked polymer substantially covered by the second biocompatible and cross-linked polymer” indicates that the second biocompatible and cross-linked polymer occupies the majority of the surface of the first biocompatible and cross-linked polymer, for example, at least about 50%, 60%, 70%, 80%, 90%, 95%, 99% of the surface of the first biocompatible and cross-linked first polymer. In an embodiment, the second biocompatible and cross-linked polymer completely covers the surface of the first biocompatible and cross-linked polymer (e.g., more than 99% of the surface of the first biocompatible and cross-linked polymer is covered with the second biocompatible and cross-linked polymer). In still another embodiment, the second biocompatible and cross-linked polymer forms a matrix into which liver organoids (which are at least partially covered with the first biocompatible and cross-linked polymer) are interspersed. In such embodiment, the liver organoids (which are at least partially covered with the first biocompatible and cross-linked polymer) can be surrounded by the second biocompatible and cross-linked matrix or can be in physical contact with another liver organoid (which is at least partially covered with the first biocompatible and cross-linked polymer). The encapsulated liver tissue can comprise a further biocompatible and cross-linked polymer to cover the second biocompatible and cross-linked polymer.

The first and second biocompatible and cross-linked polymer can be the same or different. In an embodiment, the first biocompatible and cross-linked polymer is a at least partially (and in some embodiments totally) biodegradable polymer. In combination or alternatively, the second biocompatible and cross-linked polymer is at least partially (and in some embodiments totally) resistant to biodegradation. In yet another embodiment, the first biocompatible and cross-linked polymer is a biodegradable polymer and the second biocompatible and cross-linked polymer is resistant to biodegradation. In such embodiment, the first biocompatible cross-linked polymer can be more biodegradable (e.g., less resistant to biodegradation) than the second biocompatible cross-linked polymer.

In some embodiments, the first biocompatible and cross-linked polymer comprises a plurality of liver organoids. In such embodiment, the encapsulated liver tissue can comprise at least about 50, 60, 70, 80, 90, 100, 125, 150, 175, 200, 250, 300, 350, 400, 450 or 500 liver organoids per cm². In still another embodiment, the encapsulated liver tissue can comprise at most about 500, 450, 400, 350, 300, 250, 200, 175, 150, 125, 100, 90, 80, 70, 60 or 50 liver organoids per cm². In yet another embodiment, the encapsulated liver tissue comprises between about 50, 60, 70, 80, 90, 100, 125, 150, 175, 200, 250, 300, 350, 400 or 450 and about 500, 450, 400, 350, 300, 250, 200, 175, 150, 125, 100, 90, 80, 70 or 60 liver organoids per cm². In yet another embodiment, the encapsulated liver tissue comprises between about 50 and 500 liver organoids per cm². In another embodiment, the encapsulated liver tissue comprises at least about 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400 or 2500 liver organoids per cm³. In still a further embodiment, the encapsulated liver tissue comprises at most about 2500, 2400, 2300, 2200, 2100, 2000, 1900, 1800, 1700, 1600, 1500, 1400, 1300, 1200, 1100, 1000, 950, 900, 850, 800, 750, 700, 650, 600, 550, 500, 450, 400, 350, 300 or 250 liver organoids per cm³. In still another embodiment, the encapsulated liver tissue comprises between about 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300 or 2400 and about 2500, 2400, 2300, 2200, 2100, 2000, 1900, 1800, 1700, 1600, 1500, 1400, 1300, 1200, 1100, 1000, 950, 900, 850, 800, 750, 700, 650, 600, 550, 500, 450, 400, 350 or 300 liver organoids per cm³. In still another embodiment, the encapsulated liver tissue comprises between about 250 and 2500 liver organoids per cm³.

In an embodiment, the encapsulated liver tissue in culture or when implanted in vivo is capable of expressing genes and proteins associated with hepatic, mesenchymal and optionally endothelial cells. In additional embodiment, the encapsulated liver tissue (in vitro or in vivo) is capable of producing albumin, making urea from ammonia, exhibiting CyP3A4 activity and/or metabolizing drugs (known to be metabolized by the liver, such as tacrolimus and/or rifampicin). In some embodiments, the encapsulated liver tissue is capable of producing 1, 2, 3, 4, 5, 6, 7, 8, 9, 10, 11, 12, 13, 14, 15, 16, 17, 18, 19 or 20 mg of albumin per g of liver organoids in the tissue. In another embodiment, the encapsulated liver tissue upon one or more freeze-thaw cycles is capable of expressing genes and proteins associated with hepatic, mesenchymal and optionally endothelial cells, albumin production, of making urea from ammonia, of exhibiting CyP3A4 activity and/or liver-specific metabolism of drugs (such as tacrolimus and/or rifampicin). In some embodiments after freezing, the encapsulated liver tissue is capable of producing 1, 2, 3, 4, 5, 6, 7, 8, 9, 10, 11, 12, 13, 14, 15, 16, 17, 18, 19 or 20 mg of albumin per g of liver organoids in the tissue.

Process for Making Encapsulated Liver Tissue

The process for making the encapsulated liver tissue first requires to make the liver organoid(s) and then encapsulated it (them) (at least partially) in the first biocompatible and cross-linked polymer (and optionally in the second and a further biocompatible cross-linked polymer).

The liver organoid can be made by co-culturing hepatic cells, mesenchymal cells and optionally endothelial cells (all as described above) in conditions necessary to obtain a liver organoid having (i) a cellular core comprising hepatic, mesenchymal and optionally endothelial cells, (ii) a substantially spherical shape and (iii) a relative diameter between about 50 and about 500 μm. In some embodiments, these conditions include culturing the cells in suspension (e.g., ultra-low adherent conditions) so as to promote the formation of the liver organoids.

The hepatic cells to be included in the encapsulated liver tissue can be obtained from different origins (mammals for example) and sources (primary cell culture, cell line, differentiated stem cells), provided that they have been submitted to at least one process as described herein. The hepatic cells can be from different types such as definitive endoderm cells, posterior foregut cells, cells of the hepatocyte lineage, hepatocyte-like cells and/or biliary epithelial cells. Hepatic cells from a single organoid can be from the same or different origin, from the same or different source and from the same or different type.

The mesenchymal cells to be included in the encapsulated liver tissue can be obtained from different origins (mammals for example) and sources (primary cell culture, cell line, differentiated stem cells). The mesenchymal cells can be from different types such as mesenchymal stem cells, adipocyte, muscle cells or fibroblasts. Mesenchymal cells from a single organoid can be from the same or different origin, from the same or different source and from the same or different type. In an embodiment, mesenchymal stem/progenitor cells are used. In still another embodiment, the mesenchymal stem/progenitor cells are obtained from differentiating a stem cell (such as pluripotent stem cells). In still another embodiment, the mesenchymal stem/progenitor cells are obtained from differentiating pluripotent stem cells (for example by culturing pluripotent stem cells on plastic without coating in DMEM high glucose supplemented with knock-out serum replacement). The mesenchymal cells can be used fresh or cryopreserved prior to the formation of the liver organoids.

When present, the endothelial cells to be included in the encapsulated liver tissue can be obtained from different origins (mammals for example) and sources (primary cell culture, cell line, differentiated stem cells). The endothelial cells can be from different types such as endothelial progenitor cells and endothelial cells. In an embodiment, endothelial progenitor cells are used. Endothelial cells from a single organoid can be from the same or different origin, from the same or different source and from the same or different type. In still another embodiment, the endothelial progenitor cells are obtained from differentiating a pluripotent cell (such as pluripotent stem cells). In still another embodiment, the endothelial progenitor cells are obtained from differentiating pluripotent stem cells (for example by culturing pluripotent stem cells with CHIR99021 and/or Activin A in combination with BMP4, bFGF and/or VEGF). The endothelial cells can be used fresh or cryopreserved prior to the formation of the liver organoids.

In an embodiment, the liver organoid is prepared from a single population of pluripotent stem cells. The pluripotent stem cells can be induced using methods known in the art such as viral transduction (for example by using Sendai virus system) or using a synthetic mRNA approach. The population of pluripotent stem cells can be obtained from one or more colonies of induced pluripotent stem cells (iPSCs). In the embodiment in which the liver organoid is prepared from the same population of pluripotent stem cells, the population of iPSCs is divided in at least two (and in some embodiments at least three) subpopulations each submitted to different culture conditions to generate hepatic and mesenchymal (and, in some embodiments, endothelial cells).

Once each of the different cells are obtained, they are combined and cultured in suspension to generate the liver organoid. To control the size of the liver organoids, it is possible to culture the cells in ultra-low-adherent conditions (e.g., in suspension) using micro-cavities having a diameter between 100 to 1 000 μm. In some embodiments, the micro-cavities have a diameter and depth per cm² of about 500 μm. In some embodiments, once the original liver organoids are formed, they can be cultured (for expansion) in suspension in a bioreactor. In an embodiment, the hepatic and mesenchymal are combined at a ratio, prior to culture, of 1 endodermal cells to 0.1-0.7 mesenchymal cells. In still another embodiment, when the endothelial cells are present, they are combined with endodermal cells at a ratio, prior to culture of 1 endodermal cell for of 0.2-1 endothelial cell. In still another embodiment, the ratio between the hepatic, mesenchymal and endothelial cells is 1:0.2:0.7 prior to culture. It is understood that, during culture, the ratio between the different cells may change since some are going to preferentially proliferate while other will preferentially differentiate. It is also understood that other ratios can be used to obtain the liver organoids as described herein. During the process of making the liver organoid, no physical scaffold or exogenous matrix material (other than the tissue culture vessel) is required.

The liver organoids can be used directly to make the encapsulated liver tissue. In an embodiment, the liver organoids can be cryopreserved prior to their introduction in the encapsulated liver tissue.

The polymer that can be used in the encapsulated liver tissue forms an hydrogel around the liver organoid(s). As known in the art, an hydrogel refers to polymeric chains that are hydrophilic in which water is the dispersion medium. Hydrogels can be obtained from natural or synthetic polymeric networks. In the context of the present disclosure, encapsulation within the hydrogel prevents embedded liver organoids from leaking out of the polymer, thus eliminating or reducing the risk that cells of the liver organoids could give rise to an immune reaction or a tumor within the recipient's body upon implantation.

In the context of the present disclosure, a polymer is considered “biocompatible” when is it does not exhibit toxicity towards the cells of the liver organoids or when introduced into a subject (e.g., a human for example). In the context of the present disclosure, it is preferable that the biocompatible polymer does not exhibit toxicity towards the cells of liver organoid when placed in vivo in a subject (e.g., a human for example). Hepatotoxicity can be measured, for example, by determining hepatocyte-like cells apoptotic death rate (e.g., wherein an increase in apoptosis is indicative of hepatotoxicity), transaminase levels (e.g., wherein an increase in transaminase levels is indicative of hepatotoxicity), ballooning of the hepatocyte-like cells (e.g., wherein an increase in ballooning is indicative of hepatotoxicity), microvesicular steatosis in the hepatocyte-like cells (e.g., wherein an increase in steatosis is indicative of hepatotoxicity), biliary cells death rate (e.g., wherein an increase in biliary cells death rate is indicative of hepatotoxicity), γ-glutamyl transpeptidase (GGT) levels (e.g., wherein an increase in GGT levels is indicative of hepatotoxicity). Biocompatible polymers include, but are not limited to, carbohydrates (glycosaminoglycan such as hyaluronic acid (HA), chondroitin sulphate, dermatan sulphate, keratan sulphate, heparan sulphate, alginate, chitosan, heparin, agarose, dextran, cellulose, and/or derivatives thereof), proteins (collagen, elastin, fibrin, albumin, poly (amino acid), glycoprotein, antibody and/or derivatives thereof) and/or synthetic polymers (e.g., based on poly(ethylene glycol) (PEG), poly(hydroxyethyl methacrylate) (PHEMA) and/or poly(vinyl alcohol) (PVA)).The biocompatible polymer can be a single polymer or a mixture of polymers (for example those described in US2012/01420069). Exemplary biocompatible polymers includes, but are not limited to, poly(ethylene) glycol, polylactic acid (PLA), polyglycolic acid (PGA), polycaprolactone (PCL), fibrin, polysaccharidic materials (like chitosan, proteoglycans or glycosaminoglycans (GAGs)), alginate, collagen, thiolated heparin and mixtures thereof. In some embodiments, the biocompatible polymers can be linear, branched and optionally grafted with peptides (e.g., RGD), growth factors, integrins or drugs.

In some embodiments, the polymer is “low-immunogenic polymer” and does not elicit or elicits only a minimal immune response in the recipient. This low-immunogenic polymer is also capable of masking one or more antigenic determinant of a cell and lowering or even preventing an immune response to the antigenic determinant when such an antigenic determinant is introduced into an allogeneic subject.

The polymer present in the encapsulated liver tissue of the present disclosure are preferably cross-linkable, e.g., capable of being cross-linked. The polymers can be cross-linked thermally, chemically (e.g., by using one or more peptides, such as, VPMS, RGD, etc.) or by the use of pH or light (e.g., photopolymerization, using UV light for example).

The polymers of the present disclosure can either be biodegradable (e.g., susceptible of being hydrolysed by the metabolism of a living organism) or be totally or partially resistant to biodegradation (e.g., resistant to hydrolysis when subjected to the metabolism of a living organism). Exemplary biocompatible and biodegradable polymers include, but are not limited to poly(ethylene-glycol)-maelimide (PEG-Mal) 8-arm. Exemplary biocompatible and biodegradation-resistant polymers include, but are not limited to, poly(ethylene-glycol)-vinyl sulfone (PEG-VS).

Once the liver organoids are obtained, they are contacted with the first biocompatible and cross-linkable polymer to at least partially (and in some embodiments substantially) cover the liver organoids. The polymer can be used at different concentrations. In an embodiment, the concentration of the polymer, upon contacting the liver organoids, is between about 1% and 15% (weight /volume). In an embodiment, the concentration of the polymer, upon contacting the liver organoid, is at least about 1%, 2%, 3%, 4%, 5%, 6%, 7%, 8%, 9%, 10%, 11%, 12%, 13% or 14%. In yet another embodiment, the concentration of the polymer, upon contacting the liver organoids, is equal to or lower than about 15%, 14%, 13%, 12%, 11%, 10%, 9%, 8%, 7%, 6%, 5%, 4%, 3% or 2%. Once the liver organoids have been contacted with the first polymer, the latter is cross-linked (either thermally, chemically or by using pH or light). Cross-linking the first biocompatible polymer is achieved by creating additional bonds (and in some embodiments additional covalent bonds) between different molecules of the polymer and/or within the same molecule of the polymer. In some embodiments, the cross-linking of the first biocompatible polymer will create additional bonds (and in some embodiments additional covalent bonds) between the polymeric molecules and the surface of the liver organoid. In some embodiments, the first polymer is at least partially biodegradable.

In some embodiments, the liver organoids that have been covered or encapsulated (at least partially) with the first biocompatible cross-linked polymer can be contacted with a second biocompatible cross-linkable polymer to at least partially (and in some embodiments substantially) cover the encapsulated liver tissue. Once the encapsulated liver organoids have been contacted with the second polymer, the latter is cross-linked (either thermally, chemically or by using pH or light). Cross-linking the second biocompatible polymer is achieved by creating additional bonds (and in some embodiments additional covalent bonds) between different molecules of the polymer and/or within the same molecule of the polymer. In some embodiments, the cross-linking of the second biocompatible polymer will create additional bonds (and in some embodiments additional covalent bonds) between the polymeric molecules and the first biocompatible and cross-linked polymer and, in some embodiments, the surface of the liver organoid. In some embodiments, the second polymer is, at least partially, resistant to biodegradation.

In some embodiments, the process also includes a step of contacting the encapsulated liver organoids (at least partially covered by the first/second biocompatible cross-linked polymer) with a further biocompatible and cross-linkable polymer to cover the encapsulated liver organoid. Once the liver organoids have been contacted with the further polymer, the latter is cross-linked (either thermally, chemically or by using pH or light). Cross-linking of the further biocompatible polymer is achieved by creating additional bonds (and in some embodiments additional covalent bonds) between different molecules of the polymer and/or within the same molecule of the polymer. In some embodiments, the cross-linking of the further biocompatible polymer will create additional bonds (and in some embodiments additional covalent bonds) between the polymeric molecules and the second biocompatible and cross-linked polymer and, in some embodiments, the first biocompatible and cross-linked polymer and/or the surface of the liver organoids.

The process can be designed to provide a plurality of monodispersed liver organoids within the first biocompatible and crossed-linked polymer. For example, hepatic progenitor cells, endothelial progenitor cells and mesenchymal progenitor cells can be obtained from differentiating a single iPSC. The cells can be mixed and co-cultured in suspension to form the liver organoid. In some embodiments, the cells of the hepatocyte lineage have differentiated into hepatocyte-like cells which substantially cover a cellular core formed by mesenchymal and endothelial progenitor cells (prior to the introduction of the liver organoids in the encapsulated liver tissue). In a further embodiment, the liver organoid substantially spherical in shape and has a relative diameter of about 150 μM. The liver organoids can then encapsulated, using a cross-linking agent (UV light shown for example), in a first compatible and cross-linkable matrix. The encapsulated liver tissue can be used as transplantable liver tissue (having for example, a size between 5 mm and 10 cm) in regenerative medicine. Alternatively, the liver organoids can be designed to a multiwell plate and used in drug development to determine metabolism or hepatotoxicity of screened compounds.

The process can be designed to provide a plurality of liver organoids individually covered (at least partially) with the first biocompatible and cross-linked polymer which are then incorporated in a matrix made of the second biocompatible and cross-linked polymer. In such embodiment, the plurality of liver organoids individually covered (at least partially) with the first biocompatible and cross-linked polymer are first formed and then contacted with the second biocompatible and cross-linkable polymer to be cross-linked.

The process can also be designed to provide a plurality of individual (e.g., mono-dispersed) liver organoids which are covered by the first and, optionally, the second compatible and cross-linked polymer. In such embodiment, the encapsulated liver tissue can comprise at least about 50, 60, 70, 80, 90, 100, 125, 150, 175, 200, 250, 300, 350, 400, 450 or 500 liver organoids per cm². In still another embodiment, the encapsulated liver tissue can comprise at most about 500, 450, 400, 350, 300, 250, 200, 175, 150, 125, 100, 90, 80, 70, 60 or 50 liver organoids per cm². In yet another embodiment, the encapsulated liver tissue comprises between about 50, 60, 70, 80, 90, 100, 125, 150, 175, 200, 250, 300, 350, 400 or 450 and about 500, 450, 400, 350, 300, 250, 200, 175, 150, 125, 100, 90, 80, 70 or 60 liver organoids per cm². In yet another embodiment, the encapsulated liver tissue comprises between about 50 and 500 liver organoids per cm². In another embodiment, the encapsulated liver tissue comprises at least about 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400 or 2500 liver organoids per cm³. In still a further embodiment, the encapsulated liver tissue comprises at most about 2500, 2400, 2300, 2200, 2100, 2000, 1900, 1800, 1700, 1600, 1500, 1400, 1300, 1200, 1100, 1000, 950, 900, 850, 800, 750, 700, 650, 600, 550, 500, 450, 400, 350, 300 or 250 liver organoids per cm³. In still another embodiment, the encapsulated liver tissue comprises between about 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300 or 2400 and about 2500, 2400, 2300, 2200, 2100, 2000, 1900, 1800, 1700, 1600, 1500, 1400, 1300, 1200, 1100, 1000, 950, 900, 850, 800, 750, 700, 650, 600, 550, 500, 450, 400, 350 or 300 liver organoids per cm³. In still another embodiment, the encapsulated liver tissue comprises between about 250 and 2500 liver organoids per cm³.

In an embodiment, the encapsulated liver tissue can be directly used in the therapeutic and screening methods described herein or can be cryopreserved to increase its storage time.

Therapeutic Use of the Encapsulated Liver Tissue

The encapsulated liver tissue described herein can be used as a medicine. Because it exhibits some of the biological functions of the liver and thus can be used in vivo or ex vivo to restore or improve liver functions in a subject in need thereof. Liver function can be assessed, for example, by determining the synthesis of albumin and clotting factors (e.g., fibrinogen, prothrombin, factors V, VII, VIII, IX, X, XI, XIII, as well as protein C, protein S and antithrombin), whereas an increase in the synthesis of albumin and/or clotting factors is indicative of restored or improved liver function. Liver function can also be assessed by measuring the International Normalized Ratio or INR (e.g., a decrease in INR is indicative of a restored or improved liver function). Liver function can also be assessed by measuring the detoxification of ammonia to urea (e.g., a decrease in the level of ammonia and/or an increase in the level of urea is indicative of restored or improved liver function).

In such embodiment, the encapsulated liver tissue is intended to be in contact with a biological fluid of the subject intended to be treated. In such embodiment, the encapsulated liver releases synthetized proteins and metabolites (albumin, clotting factors and/or urea) needed by the subject into the biological fluid and can even absorb toxic substances to be metabolized (ammonia, unconjugated bilirubin, cholesterol, tyrosine, etc.) from the biological fluid. The encapsulated liver tissue can be used to restore lacking/reduced enzymatic functions in inborn errors of liver metabolism.

In order to restore or improve liver functions, the encapsulated liver tissue can be grafted in vivo in the subject having reduced, little to no liver functions. As such, the encapsulated liver tissue can be, for example, implanted in the peritoneal cavity in connection with peritoneal fluids. Alternatively, the encapsulated liver tissue can be grafted on the recipient's liver, in connection with liver fluids. In yet another example, the encapsulated liver tissue can be grafted subcutaneously or intra-muscularly, in connection with lymphatic fluids or blood.

Alternative, in order to restore or improve liver functions, the encapsulated liver tissue can be used as the cellular component of an ex vivo detoxifying device (e.g., an extracorporeal device). In such embodiment, the blood and/or the peritoneal fluid of the treated subject is contacted ex vivo with the encapsulated liver tissue for providing proteins and metabolites (albumin, clotting factors and/or urea) an adsorb or metabolize potentially toxic substances (ammonia, unconjugated bilirubin, cholesterol, tyrosine, etc.).

The encapsulated liver tissue can be used with various subjects, including mammals and especially humans, who would benefit from restoring or improving liver functions. The cells of the encapsulated liver tissue can be autologous, allogeneic or xenogeneic to the subject intended to be treated. However, because the encapsulated liver tissue can be designed in order to prevent physical contact with the cells (especially the immune cells) of the intended recipient, there is no need to use autologous cells or immunosuppressive drugs to prevent immunological recognition and reaction by the intended recipient. This can be done, for example, by using an encapsulated liver tissue comprising only one biocompatible and cross-linked polymer or both a first and a second biocompatible and cross-linked polymer and/or using a low-immunogenic polymer.

In some embodiments, the encapsulated liver tissue can be designed to be manipulated and introduced into the subject by surgery, for example using a laparoscopic procedure. In addition, because the liver tissue is encapsulated in a biocompatible (and in some embodiments, low-immunogenic) polymer, it is possible to remove the encapsulated liver tissue from the subject once the liver function has been restored or the encapsulated liver tissue can no longer improve liver function.

The encapsulated liver tissue can be used to treat liver failure. Liver failure occurs when large parts of the liver become damaged beyond repair and the liver is no longer able to function. Early symptoms of liver failure include nausea, loss of appetite, fatigue and diarrhea. As the condition progresses, the following symptoms can also be observed jaundice, bleeding, swollen abdomen, mental disorientation or confusion (known as hepatic encephalopathy), sleepiness as well as coma. Liver failure can be acute, chronic or acute-on-chronic. The most common causes of chronic liver failure are non-alcoholic steatohepatitis, hepatitis B, hepatitis C, long-term alcohol consumption, cirrhosis, hemochromatosis and malnutrition. In chronic liver failure, liver cell transplantation is most often practiced via the portal circulation. However, in the case of chronic liver failure secondary to cirrhosis, the disappearance of hepatic sinusoidal fenestrations (capillarization) could prevent the injected cells injected through the portal circulation to reach the liver parenchyma and implant in the liver lobules. This could hamper the maturation and function of the transplanted cells and entail complications such as sinusoidal and portal thrombosis. Since it does not require intraportal injection or immunosuppression, the encapsulated liver tissue described herein would allow treating hundreds of thousands of patients with cirrhosis and chronic (or acute-on-chronic) liver failure, even those not eligible for transplant, preventing or reducing severe complications (hepatic encephalopathy, coagulopathy, etc.) and improving survival.

The encapsulated liver tissue described herein can also be used for treating acute liver failure. The most common causes of acute liver failure are reactions to or overdoses of prescription and herbal medicines, viral infections (including hepatitis A, B, and C), as well as ingestion of poisonous wild mushrooms, autoimmune hepatitis or Wilson disease. Acute liver failure can occur rapidly, sometimes in less than 48 hours, and is thus difficult to prevent. Furthermore in acute liver failure, liver functions are so compromised subjects need to be transplanted with fully mature and functional hepatic cells. In some embodiments, the encapsulated liver tissue can be used to treat or alleviate the symptoms of acute liver failure. The encapsulated liver tissue is either grafted in the subject in need thereof or used as an external (ex vivo) detoxifying device to treat the blood of the subject in need thereof (extracorporeal liver support, bioartificial liver device or liver dialysis). Depending on the number of liver organoids in the encapsulated liver tissue and the severity of the conditions, one or more than one encapsulated liver tissue can be used to treat the subject. The encapsulated liver tissue(s) can be used simultaneously or in sequence. When the encapsulated liver tissue is used to treat or alleviate the symptoms of liver failure, cells allogeneic to the subject to be treated can be used.

The encapsulated liver tissue can also be used to treat or alleviate the symptoms of monogenic inborn error of liver metabolism (e.g., Criggler-Najjar syndrome, familial hypercholesterolemia, urea cycle disorders such as N-acetylglutamate synthase deficiency, carbamoyl phosphate synthase deficiency, ornithine transcarbamylase deficiency, citrullinemia, argininosuccinate lyase deficiency, arginase deficiency, hereditary tyrosinemia type I, etc.). In this embodiment, the encapsulated liver tissue provides the lacking metabolic function, reducing symptoms, preventing or reducing complications and/or reducing or eliminating the need for lifelong treatments or diets.

The encapsulated liver tissue can be designed as an implantable product (for example a encapsulated liver tissue sheet) to treat acute and chronic liver failure without the need for immunosuppression. In such embodiment, the implantable tissue sheet comprises about thousands liver organoids per cm². In some embodiments, the encapsulated liver tissue sheet can be positioned within a container (such as, for example a custom-made, permeable bag) to ease manipulation and fixation to the desired site of implantation. In further embodiments, in order to be manipulated easily, the implantable tissue sheet can be at least of 1 mm-thick and, in some additional embodiments, at least 5 mm to 10 cm-wide. The encapsulated liver tissue can be made to any shape or size required and can be trimmed or cut during implementation.

Hepatic Metabolism and Hepatotoxicity Screening Methods and Kits

Since the encapsulated liver tissue described herein retains at least some hepatic function it can be used as an in vitro model to determine how an agent (such as a potential drug) is metabolized by the liver to rationalize drug discovery and development. It can also be used to determine if an agent exhibits hepatotoxicity. When administered to the general circulation, the vast majority of (suspected) therapeutic agents (approved or in development) are metabolized in some way or another by the cells of the liver. In some embodiments, the encapsulated liver tissue described herein can be used to determine the hepatotoxicity (e.g., drug-induced liver toxicity), if any, of an agent (such as a putative therapeutic agent). Drugs (approved and investigational) are an important cause of liver injury. More than 900 drugs, toxins, and herbs have been reported to cause liver injury, and drugs account for 20-40% of all instances of fulminant hepatic failure. Approximately 75% of the idiosyncratic drug reactions result in liver transplantation or death. Drug-induced hepatic injury is the most common reason cited for withdrawal of an approved drug. Determining early the hepatotoxicity profile of an agent (such as a drug) can be useful to rationalize drug discovery and development.

The encapsulated liver tissue described herein does exhibit at least some liver function and can thus be used in vitro to determine the hepatic metabolism and/or the hepatotoxicity of an agent (such as a chemical agent, a biological agent, a natural drug product or mixture). The method can be used to determine the hepatic metabolism of a single agent or a combination of agents.

In order to do so, the agent or the combination of agents to be tested is/are placed in contact with the encapsulated liver tissue so as to provide a test mixture under conditions sufficient to allow an effect of the agent on at least one (and in some embodiments, two or three) cell types of the at least one liver organoid of the encapsulated liver tissue. The test mixture comprises the agent and the encapsulated liver tissue. Then, at least one agent-related hepatic metabolite of the agent is determined in at least one (and in some embodiments, at least two or three) cell types of the at least one liver organoids of the encapsulated liver tissue or in the test mixture. As used in the context of the present disclosure, the expression “agent-related metabolite” refers to a metabolite which can be formed by hydrolyzing the agent that is being tested.

Alternatively or in combination, at least one hepatic parameter is determined in at least one (and in some embodiments, at least two or three) cell types of the at least one liver organoids of the encapsulated tissue or in the test mixture. Hepatic parameters which can be determined include, but are not limited to albumin production, urea production, ATP production, glutathione production, cytochrome P450 (CYP) metabolic activity, expression of liver-specific genes or proteins (e.g., a CYP enzyme (CyP2C9, CyP3A4, CyP1A1, CyP1A2, CyP2B6 and/or CyP2D6), responses to hepatotoxins, cellular death (e.g. by measuring lactate dehydrogenase or transaminases in the test mixture), cellular apoptosis, cellular necrosis, cellular metabolic activity (e.g. live/dead assay, caspase 3/7 assay, MTT assay or WST-1 based tests), mitochondrial function, and/or bile acid production. Once the at least one (or the plurality of) hepatic parameter has been obtained, it is compared to a corresponding control hepatic parameter. In an embodiment, the control hepatic parameter can be obtained in the absence of the screened agent (or the combination of screened agents) or in the presence of the vehicle for dissolving the screened agent (of the combination of screened agents). The determination step can be conducted on all or some of the cells of the encapsulated liver tissue. In an embodiment, the determination step is conducted on hepatocyte-like cells and/or biliary epithelial cells of the encapsulated liver tissue.

The method also includes a comparison to determine if the agent is metabolized by the liver organoids of the encapsulated liver tissue and/or if the agent exhibits hepatotoxicity towards the cells of the liver organoids of the encapsulated liver tissue. In order to do so, a comparison is made between the measured agent-related hepatic metabolite and a control agent-related hepatic metabolite. For example, the control agent-related metabolite can be the agent itself in an intact (e.g., unhydrolyzed) form. When it is determined that an agent-related metabolite which differ from the control agent-related metabolite is present, then it is determined how the agent is metabolized by the hepatic cells. A comparison can also be made between the measured hepatic parameter and a control hepatic parameter. For example, the control hepatic parameter can be obtained in the absence of the agent. When it is determined that an hepatic parameter differs from the control hepatic parameter, then it is determined if the agent exhibits hepatotoxicity.

In an embodiment, the method is used to determine if the screened agent (or the combination of screened agents) exhibits hepatotoxicity. In such embodiment, it is determined if contacting the screened agent (or the combination of screened agents) induces toxicity in at least one cell (for example an hepatocyte or a biliary epithelial cells) of the liver organoid of the encapsulated liver tissue. Toxicity can be measured, for example by determining cell death (e.g. by measuring lactate dehydrogenase or transaminases in the test mixture), cell metabolic viability (e.g. live/dead assay, caspase 3/7 assay, MTT assay or WST-1 based tests), mitochondrial function (e.g., a reduction of mitochondrial function is indicative with hepatotoxicity), modulation in the activity of one or more enzymes (such as, for example, CYP2E1) in the cytochrome P450 system (e.g., an increase in the activity of the enzyme(s) of the cytochrome P450 system is indicative of hepatotoxicity) and/or modulation in the production of bile acids (e.g., an increase in bile acid productions is indicative of hepatotoxicity). The method can include comparing the toxicity results of the screened agent with a control agent (either known not to induce hepatotoxicity or known to induce hepatotoxicity).

The method can also include contacting the screened agent (or the plurality of screened agents) against encapsulated liver tissues obtained with liver organoids having different metabolic activity. For example, liver organoids can be made using cells from different origins and sources in order to perform specific metabolic functions at different levels (thus representing variations found among individuals in the general population). For example, the obtained encapsulated liver tissues with different metabolic activity can be generated in different wells of a single plate, in order to allow testing the screened agent comparatively on each and all of them. In an embodiment, liver organoids can be derived from different genders, races and/or genotypes. The screened agent could be tested against these different genders, races and/or genotypes to determine differences in metabolism or if hepatotoxicity is present in all or only some genders, races and/or genotypes. In an embodiment, the mesenchymal and/or endothelial components of the liver organoids can be similar between the plurality of liver organoids but the hepatocyte-like cells and biliary epithelial cells are from different genders, races and/or genotypes. As an example, each different encapsulated liver tissue can be located in a different well (in multiple repetitions if necessary) and the same screened agent can be contacted with each different encapsulated liver tissue.

In some embodiments, the encapsulated liver tissue used in the screening method does not include a second or a further biocompatible cross-linked polymer and instead consists essentially of the liver organoids and the first biocompatible cross-linked polymer as described herein.

The screening method can use liver organoids which have been encapsulated individually or liver organoids which have been encapsulated in a matrix containing more than one liver organoids. In the latter, the encapsulated liver tissue can be located at the bottom of a well making it very convenient to add the screened agent and washing the encapsulated liver tissue prior to the determining step.

The present disclosure also provides a kit for determining hepatic metabolism or hepatotoxicity. The kit comprises the encapsulated liver tissue of described herein and instructions for performing the method described. In some embodiments, the kit further comprises a tissue culture support which can optionally comprises at least one well. In additional embodiments, the encapsulated liver tissue can be located at the bottom of the at least one well and, if necessary, attached (covalently or not) to the surface of the well. The kit can also comprise reagents to perform the hepatic metabolism or hepatotoxicity measurements (e.g., live/dead assay, caspase 3/7 assay, MTT assay, WST-1 assay, and/or LDH measurement for example).

The present invention will be more readily understood by referring to the following examples which are given to illustrate the invention rather than to limit its scope.

### EXAMPLE

### Production and Characterization Hepatocyte-Like Cells

Hepatocyte-like cells (HLC) were obtained from two different protocols: the protocol described herein (referred to as protocol B), a standard protocol described in PCT/CA2017/051404 (referred to as protocol A). The HLCs were then compared.

Differentiation Protocol (Protocol 8).

iPSC preparation (day −3 to 0). Three days prior to starting the differentiation, a single-cell passaging was performed using TrypLE. The iPSCs were plated on laminin-coated plates and cultured in Essential 8 Flex medium. The medium was supplemented with Revita Cell™ (ThermoFisher Scientific) for the first 24 h only. The culture medium was replaced daily.

Endoderm specification (day 1-2). Cells were washed with the culture medium DMEM/F-12 medium. Cells were then cultured in RPMI/B27 with no insulin, 1% knockout serum replacement (KOSR) supplemented with 100 ng/ml Activin A and 3 μM CHIR99021. The cells were cultured for 2 days at 37° C. in ambient O₂/5% CO₂. The culture medium was replaced daily.

Endoderm commitment (definitive endoderm, day 3-5). Cells were cultured in RPMI/B27 with no insulin, 1% knockout serum replacement supplemented with 100 ng/ml Activin A. The cells were cultured for 3 days at 37° C. in ambient O₂/5% CO₂. The culture medium was replaced daily.

Posterior foregut (day 6-10). Cells were cultured in RPMI/B27 with no insulin, 1% knockout serum replacement supplemented with 20 ng/ml BMP4, 5 ng/ml bFGF, 4 μM IWP2 and 1 μM A83-01. The cells were cultured for 5 days at 37° C. in ambient O₂/5% CO₂. The culture medium was replaced daily.

Hepatic specification (bipotent progenitor cells, day 11-15). The cells were cultured in RPMI/B27 with insulin, 2% knockout serum replacement supplemented with 20 ng/ml BMP4, 10 ng/ml bFGF, 20 ng/ml HGF and 3 μM CHIR99021. The cells were cultured for 5 days at 37° C. in ambient O₂/5% CO₂. The culture medium was replaced daily.

Hepatic maturation 1 (immature hepatocyte-like cells, day 16-20:). The cells were cultured in HBM/HCM medium (without EGF, Lonza), 1% knockout serum replacement supplemented with 20 ng/ml HGF, 3 μM CHIR99021, 20 ng/ml BMP4, 10 ng/ml bFGF, 20 ng/ml OSM, 10 μM dexamethasone and 1 μM A83-01. The cells were cultured for 5 days at 37° C. in ambient O₂/5% CO₂. The culture medium was replaced daily. Comparable results have been obtained using RPMI/B27 with insulin, 2% knockout serum replacement instead of the HBM/HCM medium (data not shown).

Hepatic maturation 2 (immature hepatocyte-like cells, day 21-25). The cells were cultured in HBM/HCM medium (without EGF, Lonza), 1% knockout serum replacement supplemented with 20 ng/ml OSM 10 μM dexamethasone. The cells were cultured for 5 days at 37° C. in ambient O₂/5% CO₂. The culture medium was replaced daily. Comparable results have been obtained using William's E medium supplemented with 1% knockout serum replacement and Primary Hepatocyte Maintenance Supplement™ (ThermoFisher Scientific) instead of HBM/HCM medium (data not shown).

Hepatic maturation 3 (mature hepatocyte-like cells, day 25-30). The cells were cultured in HBM/HCM medium (without EGF, Lonza), 1% knockout serum replacement supplemented with 10 μM dexamethasone. The cells were cultured for 5 days at 37° C. in ambient O₂/5% CO₂. The culture medium was replaced every other day. Comparable results have been obtained using William's E medium supplemented with 1% knockout serum replacement and Primary Hepatocyte Maintenance Supplement™ (ThermoFisher Scientific) instead of HBM/HCM medium (data not shown).

TABLE 1
Details for the two protocols for obtaining
hepatocyte-like cells compared in this Example.

| Time- | | | |
|---|---|---|---|
| line | Step | Protocol A | Protocol B |
| −3-0 | Plating | Essential 8 Flex | Essential 8 Flex |
| | cells for | 4% O₂, 5% CO₂ | 4% O₂, 5% CO₂ |
| | differ- | Revita Cell ™ (for | Revita Cell ™ (for |
| | entiation | the first 24 h) | the first 24 h) |
| 1-2 | Endoderm | RPMI/B27 minus | RPMI/B27 minus |
| | specification | insulin, 1% KOSR | insulin, 1% KOSR |
| | | Ambient O₂, 5% CO₂ | Ambient O₂, 5% CO₂ |
| | | Activin A (100 ng/ml) | Activin A (100 ng/ml) |
| | | CHIR99021 (3 μM) | CHIR99021 (3 μM) |
| 3-5 | Endoderm | RPMI/B27 minus | RPMI/B27 minus |
| | commitment | insulin, 1% KOSR | insulin, 1% KOSR |
| | | Ambient O₂, 5% CO₂ | Ambient O₂, 5% CO₂ |
| | | Activin A (100 ng/ml) | Activin A (100 ng/ml) |
| 6-10 | Posterior | RPMI/B27 with | RPMI/B27 minus |
| | foregut | insulin, 2% KOSR | insulin, 1% KOSR |
| | | Ambient O₂, 5% CO₂ | Ambient O₂, 5% CO₂ |
| | | BMP4 (20 ng/ml) | IWP2 (4 μM) |
| | | bFGF (10 ng/ml) | A83-01 (1 μM) |
| | | | BMP4 (20 ng/ml) |
| | | | bFGF (5 ng/ml) |
| 11-15 | Hepatic | RPMI/B27 with | RPMI/B27 with |
| | specification | insulin, 2% KOSR | insulin, 2% KOSR |
| | | Ambient O₂, 5% CO₂ | Ambient O₂, 5% CO₂ |
| | | HGF (20 ng/ml) | CHIR99021 (3 μM) |
| | | | BMP4 (20 ng/ml) |
| | | | bFGF (10 ng/ml) |
| | | | HGF (20 ng/ml) |
| 16-20 | Hepatic | William's E | HBM/HCM (without |
| | maturation 1 | medium/primary | EGF) 1% KOSR |
| | | hepatocytes | |
| | | supplement, 1% | |
| | | KOSR | |
| | | Ambient O₂, 5% CO₂ | Ambient O₂, 5% CO₂ |
| | | OSM (20 ng/ml) | CHIR99021 (3 μM) |
| | | Dexamethasone | BMP4 (20 ng/ml) |
| | | (10 μM) | bFGF (10 ng/ml) |
| | | | HGF (20 ng/ml) |
| | | | A83-01 (1 μM) |
| | | | Oncostatin M (OSM) |
| | | | (20 ng/ml) |
| | | | Dexamethasone |
| | | | (10 μM) |
| 21-25 | Hepatic | William's E | HBM/HCM (without |
| | maturation 2 | medium/primary | EGF) 1% KOSR |
| | | hepatocytes | |
| | | supplement, 1% | |
| | | KOSR | |
| | | Ambient O₂, 5% CO₂ | Ambient O₂, 5% CO₂ |
| | | OSM (20 ng/ml) | OSM (20 ng/ml) |
| | | Dexamethasone | dexamethasone |
| | | (10 μM) | (10 μM) |
| 26-30 | Hepatocyte | Not performed | HBM/HCM (without |
| | maturation 3 | | EGF) 1% KOSR |
| | | | Ambien O₂, 5% CO₂ |
| | | | Dexamethasone |
| | | | (10 μM) |

Cellular microscopy. Live cells during at the end of the differentiation process were observed to study morphology using phase contrast microscopy (EVOS FL Cell Imaging System, Thermo Fisher Scientific).

Cellular count. The cells were recovered from the culture plates using TrypLE and counted using an automated cell counter Countess II FL Automated Cell Counter, Thermo Fisher Scientific.

Immunofluorescence. The cells were fixed in 4% Paraformaldehyde and permeabilazed in 0.2% Triton X-100 for 5 min at room temperature. Nonspecific sites were blocked by incubating the cells with a 3% blocking serum (corresponding with antibody) solution for 30 min at room temperature. The fixed and permeabilized cells were then incubated with primary antibody solution (antibodies are diluted in PBS-BSA 2%) for 1 h at room temperature. The cells were incubated with secondary labelled antibody solution (fluorescence) for 30 min at room temperature protected from the light. During the last 15 min of incubation with the secondary labelled antibody, a dye (Pureblue nuclei staining, BioRad) was added to stain the nuclei. The cells were then mounted with an antifade reagent (ProLong Gold). Fluorescence was analyzed the day after the procedure. The following antibodies were used: Anti-human SOX17 dilution 1:100 from ABCAM, Anti-human FOXA2 dilution 1:100 from ABCAM; Anti-human CXCR4 dilution 1:100 from ABCAM; Anti-human AFP dilution 1:100 from DAKO; Anti-human albumin (ALB) dilution 1:100 from DAKO; anti-human CK19 dilution 1:100 from ABCAM and anti-human CK7 dilution 1:200 from ABCAM.

FACS analysis. A total of 0.5-1×10⁶ cells were aliquoted into each assay tube. Cells were stained with 100 pl of fluorochrome-conjugated primary antibody solution (membrane antigen) for 20 min at room temperature and protect from the light. Cells were subsequently fixed with 4% paraformaldehyde for 10 min at room temperature. Cells were permeabilized with 1% Triton X-100. Cells were stained with 100 pl of fluorochrome-conjugated antibody solution (intracellular antigen) and incubated in the dark at room temperature for 20 min. Cells were resuspended in 0.5 ml PBS-BSA 1%, kept at 4° C. and analyzed. The following antibodies were used for the FACS: Per-CP-Cy 5.5 anti-human SOX17 (BD Bioscience), APC anti-human CD184 (CXCR4) (BD Bioscience), PE anti human FOXA2 (BD Bioscience), PE anti-human EpCAM (BD Bioscience), APC anti-human albumin (R&D system), FITC anti-human TRA1-60 (BD Bioscience), Alexa 647 anti-human Nanog (BD Bioscience), APC anti-human Brachyury (Bio-Techne) and PerCP-Cy 5.5 anti-human c-Kit (CD117) (BD Bioscience).

Real-time RT-PCR. Total RNA was extracted (Rneasy Plus Mini Kit, Qiagen) from cultured cells to use as a template for synthesis of single-stranded cDNA. Reverse transcription was performed to obtain cDNA. The PCR reaction mix was prepared and afterwards loaded in the plate. The plate was sealed, centrifuged and then loaded into the instrument. The standard TaqMan qPCR reaction conditions were used. Data was analysed using the comparative CT (ΔΔCT) method for calculating relative quantitation of gene expression. The following TaqMan gene expression assays (from Thermo Fisher scientific) were used: Hs1053049_S1 SOX2 Taqman gene expression assay, Hs00751752_S1 SOX17 Taqman gene expression assay, Hs00171403_M1 GATA4 Taqman gene expression assay, Hs002230853_M1 HNF4A Taqman gene expression assay, Hs00173490_M1 AFP Taqman gene expression assay, Hs00609411_M1 Albumin Taqman gene expression assay, Hs99999905_M1 GAPDH Taqman gene expression assay, Hs04187555_m1 FOXA1 Taqman gene expression assay, Hs00242160 m1 HHEX Taqman gene expression assay, Hs00236830 m1 PDX1 Taqman gene expression assay, Hs00232764 m1 FOXA2 Taqman gene expression assay, Hs01005019_m1 ASGR1 Taqman gene expression assay, Hs00173490 AFP Taqman gene expression assay, Hs00607978 s1 CXCR4 Taqman gene expression assay, Hs00761767_s1 KRT19 Taqman gene expression assay, Hs00559840_m1 KRT7 Taqman gene expression assay and Hs00944626_m1 TAT Taqman gene expression assay.

Cyp 3A4 activity. Cyp3A4 activity was evaluated using “P450-Glo™ Assays” from Promega, according to manufacturer's instructions.

Urea synthesis. Urea synthesis was measured using “Quantichrom urea assay kit” from Centaur, according to manufacturer's instructions.

Albumin production. Albumin production was evaluated with “Albumin human ELISA kit” from Abcam, according to manufacturer's instructions.

Mitochondrial respiration analysis. Mitochondrial stress testing was carried out using a Seahorse Bioscience XF96 analyser (Seahorse Bioscience Inc.) in 96-well plates at 37° C. as per the manufacturer' s instructions with minor modifications. Briefly, cells were seeded at 1×10⁵ cells/well and pre-treated with different doses of acetaminophen (APAP—2, 4, 8 mM) and amiodarone (AMIO—2, 4, 8, 19 μM) 24 h prior to the assay. On the test day, the growth media was removed, washed twice and replaced with XF assay media (unbuffered DMEM, d5030 Sigma, 25 mM glucose, 2 mM glutamine, 1 mM sodium pyruvate, pH 7.4) and the plate was incubated in a CO₂-free incubator for 1 h at 37° C. The hydrated cartridge sensor was loaded with the appropriate volume of mitochondrial modulators to achieve final concentrations in each well: oligomycin (2 μM), carbonilcyanide p-triflouromethoxyphenylhydrazone (FCCP) (2 μM) and with rotenone/antimycin A (both 1 μM). Then, levels of basal respiration, ATP production, proton leak, maximal respiration and non-mitochondrial respiration were analyzed from the OCR values as described in manufacturers protocol.

TABLE 2
Abbreviations used.

| | |
|---|---|
| iPSC | Non-differentiated pluripotent stem cells |
| DE | Endodermal cell at day 5 of the |
| | differentiation protocol |
| PFG | Posterior foregut cells obtained at day |
| | 10 of the differentiation protocol |
| HB | Hepatic progenitor cells obtained at day |
| | 15 of the differentiation protocol |
| FPHH | Freshly isolated primary human fetal |
| | hepatocytes |
| PHH | Primary human hepatocytes (adult) |
| HLC | Hepatocyte-like cells that we obtain at |
| | the end of the differentiation protocol. |
| HLC-A | Hepatocyte-like cells obtained with the |
| | standard differentiation protocol |
| | (protocol A) |
| HLC-B | Hepatocyte-like cells obtained with the |
| | differentiation protocol of protocol B |

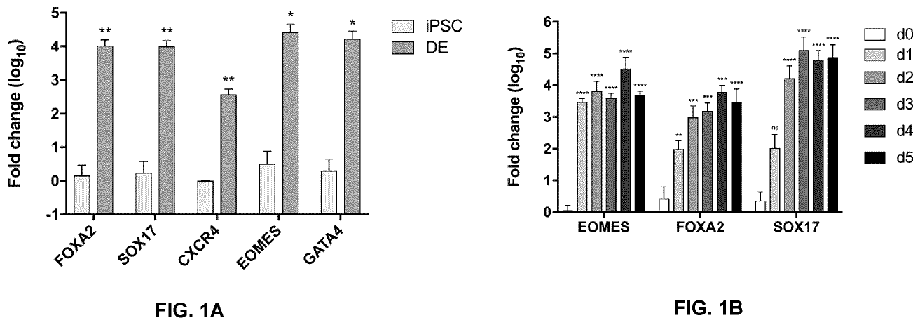

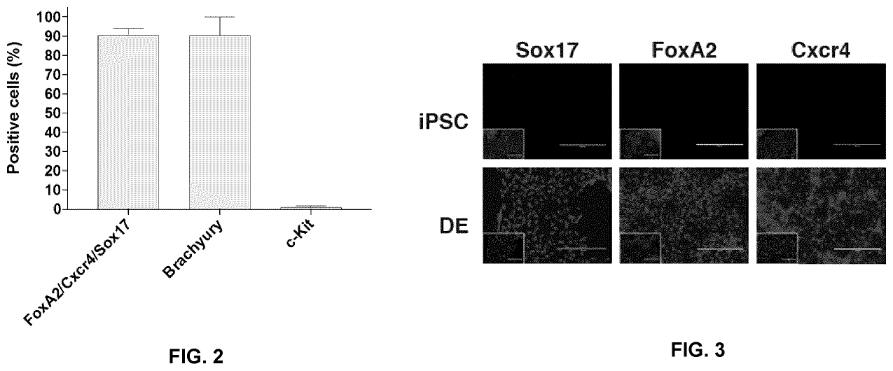

Five days of endoderm induction treatment of hiPSCs resulted in a homogenous monolayer of cells expressing specific endoderm markers SOX17, FOXA2, GATA4, CXCR4 and EOMES (FIG. 1). The homogeneity of the population has been confirmed by flow cytometry analysis which showed that more than 80% of the cells were triple positive for SOX17, FOXA2 and CXCR4 and that the cells do not express c-Kit (FIG. 2). Immunostaining revealed that most of the cells were positive for the definitive endoderm markers SOX17, FOXA2 and CXCR4 (FIG. 3—bottom panel). Similar results have been obtained by differentiating human embryonic stem cells (hESCs, data not shown) instead of iPSCs.

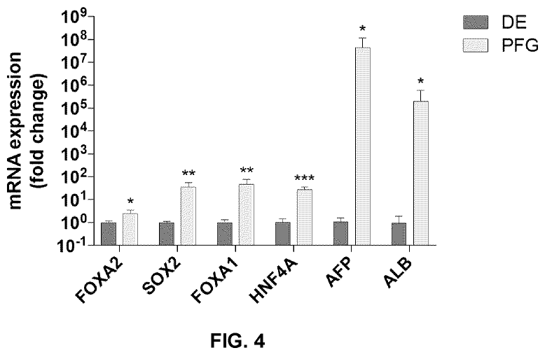

Following the endodermal induction, cells were treated for five days to induce differentiation into posterior foregut. At that stage, signals such as FGF-2 and BMP4, normally emanate from the cardiac mesoderm, were provided. In addition, the Wnt/β-catenin and TGFβ signaling pathways were inhibited (by respectively using IWP2 and A83-01) to allow expression of Hex and Prox1. As shown on FIG. 4, the cells increased their expression in foregut specific markers FOXA2, SOX2, FOXA1, HNF4A, AFP and albumin.

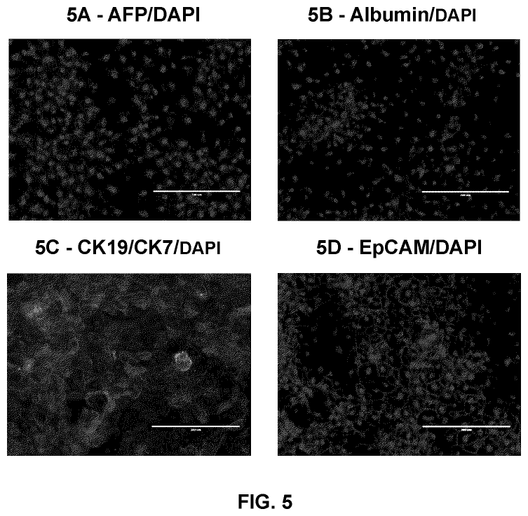

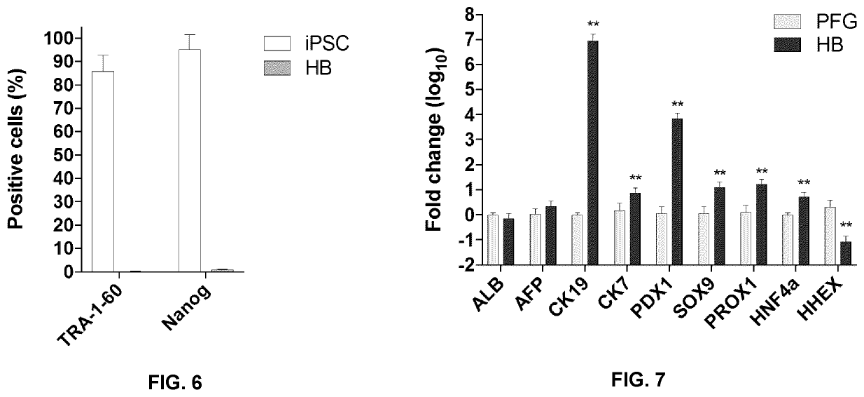

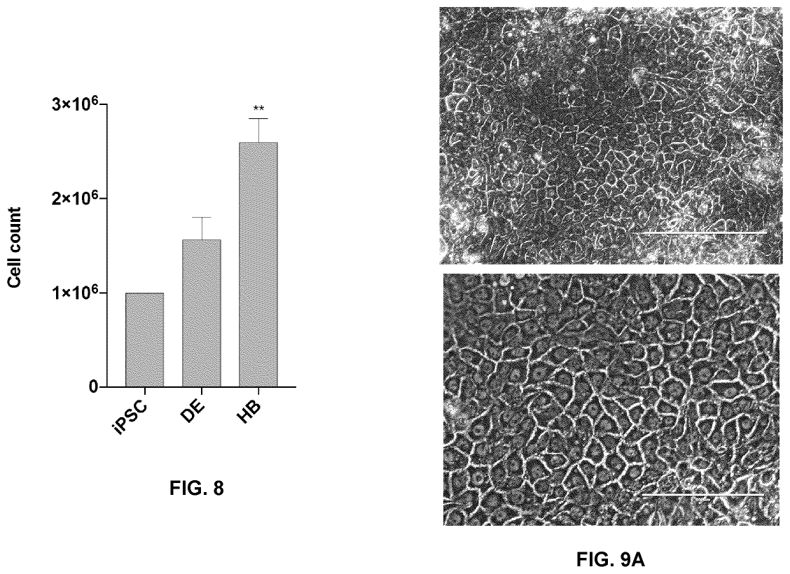

Subsequently, hepatic specification was induced (hepatoblasts with a polygonal morphology) for 5 days by maintaining the FGF-2 and BMP4 signals, adding HGF, and activating the Wnt pathway (by using CHIR99021) for promoting liver outgrowth. The cells were shown to express hepatic specific markers AFP, albumin, CK19, CK7 and EpCAM (FIG. 5). It was also determined that iPSC-derived hepatic progenitor cell population did not include undifferentiated cells (FIG. 6). RT-qPCR showed the expression of characteristic hepatoblast/hepatocyte markers such as albumin, AFP, AFP, CK19, CK7, PDX1, SOX9, PROX1, HNF4a and HHEX (FIG. 7). As shown on FIG. 8, hepatic progenitor cells showed a significant increase in cell yield when compare to endodermal cells or undifferentiated iPSCs.

To further define the hepatic commitment, TGFβ signaling was inhibited (to avoid biliary cells, by using A83-01) and the Wnt pathway was activated (by using CHIR99021). FGF-2, BMP4, HGF, OSM and dexamethasone were included. For the final stage of differentiation, OSM was removed (since after birth, hematopoiesis no longer occurs in the liver) and dexamethasone was maintained.

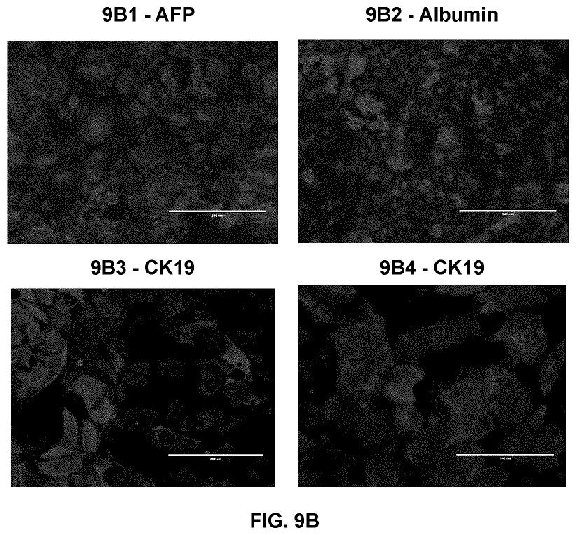

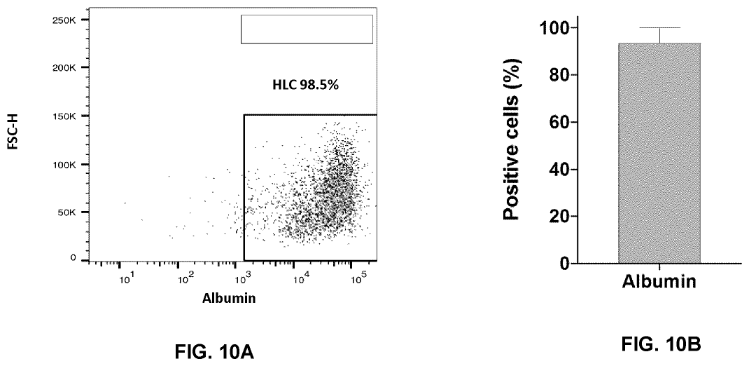

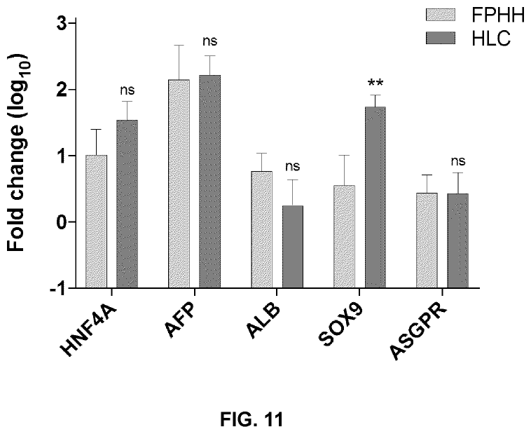

In the course of differentiation, the cell population progressively acquired the typical morphology of the hepatocyte-like cells with a large cytoplasmic to nuclear ratio, numerous vacuoles and vesicles, and prominent nucleoli. Several cells were found to be binucleated (FIG. 9A). The cells were also shown to express AFP, albumin as well as CK19 (FIG. 9B). Immunofluorescence showed and increased expression of albumin and decreased expression of AFP and CK19 in comparison to the hepatoblast stage (FIG. 9B and data not shown). Most of the cells (98.5%) were positive for albumin, as assessed by flow cytometry analysis (FIG. 10). RT-qPCR analysis showed the expression of specific hepatic genes such as albumin, AFP, HNF4a, ASGR1 and SOX9 are similar between HLC and FPHH (FIG. 11).

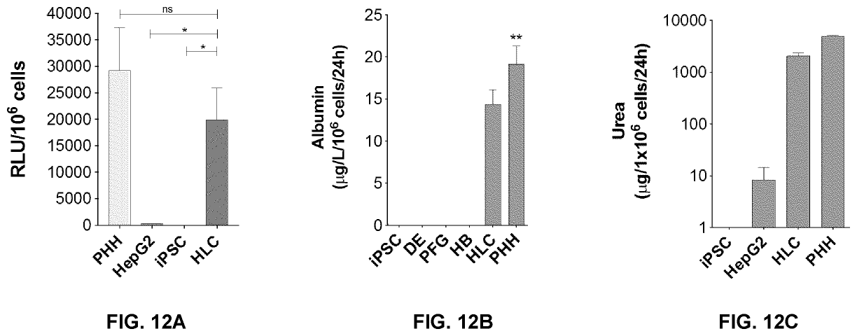

FIG. 12 compares the HLC obtained from protocol B, with primary human hepatocytes HepG2, undifferentiated iPSCs, DE cells or PFG cells. These results to show that HLC-B and FPHH have a similar CyP3A4 activity (FIG. 12A) and urea production (FIG. 12C). HLC-B cells produce less but comparable levels of albumin when compared to adult hepatocytes (FIG. 12B).

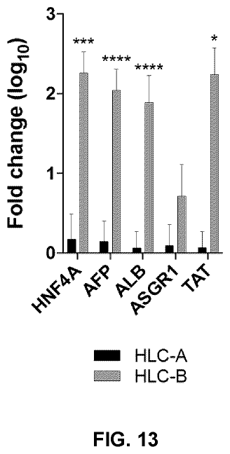

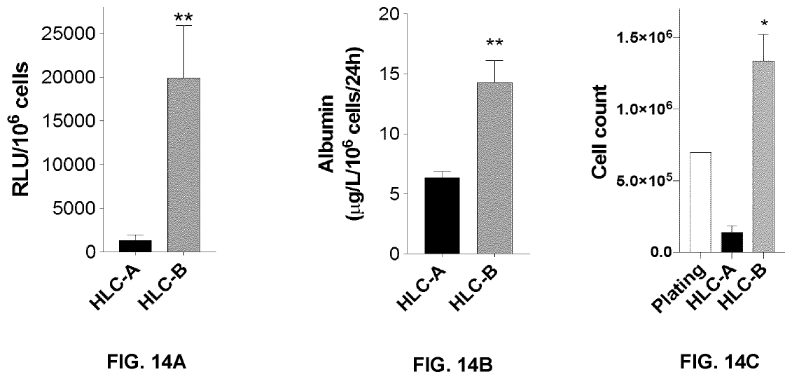

HLCs obtained from protocol B have shown to achieve a significant more important degree of differentiation in comparison to the HLCs obtained from protocol A as shown by a higher expression of the liver markers (FIG. 13), a significant higher CyP3a4 activity (FIG. 14A), albumin production (FIG. 14B) and cell yield (FIG. 14C).

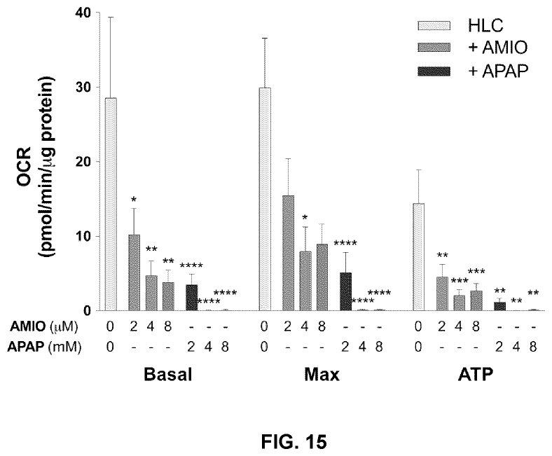

The metabolic function of the hepatocyte-like cells (obtained using protocol B), mitochondrial respiratory capacity and ATP-linked respiration were assessed in basal conditions and after increasing doses of acetaminophen (APAP) and amiodarone (AMIO), drugs specifically metabolized by the liver (FIG. 15). The results presented on Example 15 show that, in contact with the drugs, the HLCs obtained from protocol B modulate their respiration and are thus metabolically active.

While the invention has been described in connection with specific embodiments thereof, it will be understood that the scope of the claims should not be limited by the preferred embodiments set forth in the examples, but should be given the broadest interpretation consistent with the description as a whole.

1. A process of making posterior foregut cells from endodermal cells, the process comprising contacting the endodermal cells with a first culture medium excluding insulin and comprising a first set of additives under conditions allowing the differentiation of the endodermal cells into the posterior foregut cells, wherein the first set of additives excludes insulin and comprises or consists essentially of:
an activator of a bone morphogenetic protein (BMP) signaling pathway;
an activator of a fibroblast growth factor (FGF) signaling pathway;
an inhibitor of a Wnt signaling pathway; and
an inhibitor of a transforming growth factor β (TGFβ) signaling pathway.

2. The process of claim 1, further comprising making hepatic progenitor cells from the posterior foregut cells and making hepatocyte-like cells from hepatic progenitor cells.

3. The process of claim 1, wherein the first culture medium comprises serum

4. The process of claim 1, wherein the activator of the BMP signaling pathway is a BMP receptor agonist.

5. The process of claim 4, wherein the BMP receptor agonist is BMP4.

6. The process of claim 1, wherein the activator of the FGF signaling pathway is a FGF receptor agonist.

7. The process of claim 6, wherein the FGF receptor agonist is basic FGF.

8. The process of claim 1, wherein the inhibitor of the Wnt signaling pathway is capable of inhibiting the biological activity of Porcupine.

9. The process of claim 8, wherein the inhibitor of the Wnt signaling pathway is IWP2.

10. The process of claim 1, wherein the inhibitor of the TGFβ signaling pathway is capable of inhibiting the biological activity of at least one of ALK4, ALK5 or ALK7.

11. The process of claim 10, wherein the inhibitor of the TGFβ signaling pathway is A83-01.

12. The process of claim 1, wherein the endodermal cells express at least one of SOX17, GATA4, FOXA2, CXCR4 or EOMES.

13. The process of claim 1, wherein the endodermal cells fail to substantially express c-Kit.

14. The process of claim 1, wherein the posterior foregut cells express at least one of SOX2, FOXA1, FOXA2, HNF4a, AFP or albumin.

15. A population of posterior foregut cells obtainable or obtained by the process of claim 1.

16.-50. (canceled)

51. A process for making hepatic progenitor cells from endodermal cells, the process comprising or consisting essentially of:
(a) performing the process of claim 1 to obtain posterior foregut cells; and
(b) contacting the posterior foregut cells with a second culture medium comprising a second set of additives under conditions allowing the differentiation of the posterior foregut cells into the hepatic progenitor cells, wherein the second set of additives comprises or consists essentially of:
an activator of an insulin signaling pathway;
an activator of a bone morphogenetic protein (BMP) signaling pathway;
an activator of a fibroblast growth factor (FGF) signaling pathway;
an activator of an hepatocyte growth factor (HGF) signaling pathway; and
an activator of a Wnt signaling pathway.

52.-54. (canceled)

55. A process for making hepatocyte-like cells from endodermal cells, the process comprising or consisting essentially of:
(a) performing the process of claim 1 to obtain posterior foregut cells;
(b) contacting the posterior foregut cells with a second culture medium comprising a second set of additives under conditions allowing the differentiation of the posterior foregut cells into the hepatic progenitor cells, wherein the second set of additives comprises or consists essentially of:
an activator of an insulin signaling pathway;
an activator of a bone morphogenetic protein (BMP) signaling pathway;
an activator of a fibroblast growth factor (FGF) signaling pathway;
an activator of an hepatocyte growth factor (HGF) signaling pathway; and
an activator of a Wnt signaling pathway; and
(c) contacting the hepatic progenitor cells with a third culture medium comprising a third set of additives under conditions to obtain cells of the hepatocyte lineage, wherein the third set of additives comprises or consists essentially of:
an activator of an insulin signaling pathway,
an activator of a bone morphogenetic protein (BMP) signaling pathway,
an activator of a fibroblast growth factor (FGF) signaling pathway,
an activator of a hepatocyte growth factor (HGF) signaling pathway,
an activator of a Wnt signaling pathway,
an inhibitor of a transforming growth factor β (TGFβ) signaling pathway,
a cytokine, and
a glucocorticoid;
(d) contacting the cells of the hepatocyte lineage with a fourth culture medium comprising a fourth set of additives under conditions to obtain immature hepatocyte-like cells, wherein the fourth set of additives comprises or consists essentially of:
a cytokine, and
a glucocorticoid; and
(e) contacting the immature hepatocyte-like cells with a fifth culture medium excluding cytokines comprising a fifth set of additives under conditions to obtain the mature hepatocyte-like cells, wherein the fifth set of additives excludes cytokines and comprises or consists essentially of a glucocorticoid.

56. (canceled)

57. A process for making an encapsulated liver tissue, the process comprising:
(a) providing a population of hepatocyte-like cells obtained by the process of claim 55;
(b) combining and culturing, in suspension, the hepatocyte-like cells, mesenchymal and optionally endothelial cells so as to obtain at least one liver organoid comprising (i) a cellular core comprising mesenchymal and optionally endothelial cells, wherein the cellular core at least partially covered with hepatocyte-like cells and/or biliary epithelial cells, (ii) having a spherical shape and (iii) having a relative diameter between about 50 and about 500 μm; and
(c) at least partially covering the at least one liver organoid with a first biocompatible cross-linked polymer.

58.-86. (canceled)
